(19) United States
(12) Patent Application Publication
(10) Pub. No.: US 2011/0104664 A1
(43) Pub. Date: May 5, 2011

(54) BIOMARKERS AND METHODS FOR DETERMINING SENSITIVITY TO MICORTUBULE-STABILIZING AGENTS

(75) Inventors: Hyerim Lee, New York, NY (US); Edwin A. Clark, Hopkinton, MA (US); Shujian Wu, Langhorne, PA (US); Li-An Xu, Branchburg, NJ (US)

(73) Assignee: Bristol-Myers Squibb Company

(21) Appl. No.: 12/225,776

(22) PCT Filed: Mar. 30, 2007
(86) PCT No.: PCT/US2007/008269

§ 371 Date: Dec. 20, 2010

Related U.S. Application Data
(60) Provisional application No. 60/788,464, filed on Mar. 31, 2006.

(51) Int. Cl. C12Q 1/68; G01N 33/53

(52) U.S. Cl. 435/6; 435/7.1

(57) ABSTRACT

Biomarkers that are useful for identifying a mammal that will respond therapeutically or is responding therapeutically to a method of treating cancer that comprises administering a microtubule-stabilizing agent. In one aspect, the cancer is breast cancer, and the microtubule-stabilizing agent is an epothilone or analog or derivative thereof, or ixabepilone.

### FIELD OF TEE INVENTION

The present invention relates generally to the field of pharmacogenomics, and more specifically to methods and procedures to determine drug sensitivity in patients to allow the identification of individualized genetic profiles which will aid in treating diseases and disorders.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., Current Opinion in Biotechnology, 11:602-609 (2000)).

The ability to predict drug sensitivity inpatients is particularly challenging because drug responses reflect not only properties intrinsic to the target cells, but also a host's metabolic properties. Efforts to use genetic information to predict drug sensitivity have primarily focused on individual genes that have broad effects, such as the multidrug resistance genes, mdr1 and mrp1 (P. Sonneveld, J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (J. Khan et al., Cancer Res., 58:5009-5013 (1998); A. A. Alizadeh et al., Nature, 403:503-511 (2000); M. Bittner et al., Nature, 406:536-540 (2000); J. Khan et al., Nature Medicine, 7(6):673-679 (2001); T. R. Golub et al., Science, 286:531-537 (1999); U. Alon et al., P.N.A.S. USA, 96:6745-6750 (1999)). Such technologies and molecular tools have made it possible to monitor the expression level of a large number of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588 to Ashby et al.).

Recent studies demonstrate that gene expression information generated by microarray analysis of human tumors can predict clinical outcome (L. J. van't Veer et al., Nature, 415:530-536 (2002); T. Sorlie et al., P.N.A.S. USA, 98:10869-10874 (2001); M. Shipp et al., Nature Medicine, 8(1):68-74 (2002); G. Glinsky et al., The Journal of Clin. Invest., 113(6):913-923 (2004)). These findings bring hope that cancer treatment will be vastly improved by better predicting the response of individual tumors to therapy.

Needed are new and alternative methods and procedures to determine drug sensitivity in patients to allow the development of individualized genetic profiles which are necessary to treat diseases and disorders based on patient response at a molecular level.

### SUMMARY OF THE INVENTION

The invention provides methods and procedures for determining patient sensitivity to one or more microtubule-stabilizing agents. The invention also provides methods of determining or predicting whether an individual requiring therapy for a disease state such as cancer will or will not respond to treatment, prior to administration of the treatment, wherein the treatment comprises administration of one or more microtubule-stabilizing agents.

A method for identifying a mammal that will respond therapeutically to a method of treating cancer comprising administering a microtubule-stabilizing agent, wherein the method comprises: (a) exposing a biological sample from the mammal to said agent; (b) following the exposing of step (a), measuring in said biological sample the level of the at least one biomarker selected from the biomarkers of Table 2 and Table 3, wherein a difference in the level of the at least one biomarker measured in step (b), compared to the level of the at least one biomarker in a mammal that has not been exposed to said agent, indicates that the mammal will respond therapeutically to said method of treating cancer.

In another aspect, the invention provides a method for determining whether a mammal is responding therapeutically to a microtubule-stabilizing agent, comprising: (a) exposing a biological sample from the mammal to said agent; (b) following the exposing of step (a), measuring in said biological sample the level of the at least one biomarker selected from the biomarkers of Table 2 and Table 3, wherein a difference in the level of the at least one biomarker measured in step (b), compared to the level of the at least one biomarker in a mammal that has not been exposed to said agent, indicates that the mammal will respond therapeutically to said method of treating cancer.

A method for predicting whether a mammal will respond therapeutically to a method of treating cancer comprising administering a microtubule-stabilizing agent, wherein the method comprises: (a) measuring in the mammal the level of at least one biomarker selected from the biomarkers of Table 2 and Table 3; (b) exposing a biological sample from said mammal to said agent; (c) following the exposing of step (b), measuring in said biological sample the level of the at least one biomarker, wherein a difference in the level of the at least one biomarker measured in step (c) compared to the level of the at least one biomarker measured in step (a) indicates that the mammal will respond therapeutically to said method of treating cancer

In another aspect, the invention provides a method for determining whether an agent stabilizes microtubules and has cytotoxic activity against rapidly proliferating cells, such as, tumor cells or other hyperproliferative cellular disease in a mammal, comprising: (a) exposing the mammal to the agent; and (b) following the exposing of step (a), measuring in the mammal the level of at least one biomarker selected from the biomarkers of Table 2 and Table 3.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The amount of increase in the level of the at least one biomarker measured in the practice of the invention can be readily determined by one skilled in the art. In one aspect, the increase in the level of a biomarker is at least a two-fold difference, at least a three-fold difference, or at least a four-fold difference in the level of the biomarker.

The mammal can be, for example, a human, rat, mouse, dog, rabbit, pig sheep, cow, horse, cat, primate, or monkey.

The method of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the biomarker(s).

The invention also provides an isolated biomarker selected from the biomarkers of Table 2 and Table 3. The biomarkers of the invention comprise sequences selected from the nucleotide and amino acid sequences provided in Table 2 and Table 3 and the Sequence Listing, as well as fragments and variants thereof.

The invention also provides a biomarker set comprising two or more biomarkers selected from the biomarkers of Table 2 and Table 3.

The invention also provides kits for determining or predicting whether a patient would be susceptible or resistant to a treatment that comprises one or more microtubule-stabilizing agents. The patient may have a cancer or tumor such as, for example, a breast cancer or tumor.

In one aspect, the kit comprises a suitable container that comprises one or more specialized microarrays of the invention, one or more microtubule-stabilizing agents for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers selected from the biomarkers of Table 2 and Table 3.

In yet another aspect, the invention provides a kit comprising at least one of an antibody and a nucleic acid for detecting the presence of at least one of the biomarkers selected from the biomarkers of Table 2 and Table 3. In one aspect, the kit further comprises instructions for determining whether or not a mammal will respond therapeutically to a method of treating cancer comprising administering a microtubule-stabilizing agent.

The invention also provides screening assays for determining if a patient will be susceptible or resistant to treatment with one or more microtubule-stabilizing agents.

The invention also provides a method of monitoring the treatment of a patient having a disease, wherein said disease is treated by a method comprising administering one or more microtubule-stabilizing agents.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with either sensitivity or resistance to one or more microtubule-stabilizing agents.

The invention also provides antibodies, including polyclonal or monoclonal, directed against one or more biomarkers of the invention.

The invention will be better understood upon a reading of the detailed description of the invention when considered in connection with the accompanying figures.

### BRIEF DESCRIPTION OF THE FIGURES

FIG. 1 illustrates GSEA analysis of ixabepilone responders vs. non-responders populations. Blue box: down-regulated genes and red box: up-regulated genes. In the pathway enrichment analysis: all well-known pathways (>400) were investigated; this analysis is pathway centric rather than gene centric (a large list of differentially expressed genes can be mapped to a much smaller list of differentially expressed pathways to ease further analysis); and signal to noise was used instead of t-test to minimize the heterogeneity of gene expression.

FIG. 2 illustrates pathway enrichment analysis and the corresponding p values. Gene set within cell cycle pathway was the most significant enriched network in the ixabepilone responding cases compared to the non-responding cases.

FIG. 3 illustrates the top 100 genes identified through GSEA uploaded onto the Ingenuity system. The most significant network is showed here. Genes highlight with a circle were considered the hubs of the regulation network. Genes highlighted with arrows are potential predictors for ixabepilone sensitivity. Red: up-regulated in ixabepilone responders. Green: down-regulated in ixabepilone responders. There are at least four important pathways: estrogen (ESR1); ERBB2-EGFR family; p53 tumor suppressor; and E2F transcription factor.

FIG. 4 illustrates scatter plots showing the relationship between the expression level of ER and E2F1 or E2F3 among the 134 breast cancer samples.

FIG. 5 illustrates a heatmap showing the expression levels of cell cycle genes with the increasing level of Her2 in the 134 breast cancer samples. The black boxes indicate high expression and white boxes indicate low expression on the heatmap.

FIG. 6 illustrates a heatmap showing the expression levels of cell cycle genes with the increasing level of ER in the 134 breast cancer samples. The black boxes indicate high expression and white boxes indicate low expression on the heatmap.

FIG. 7 illustrates GSEA results wherein a common cis-element was found to be shared by certain cell cycle related genes.

FIG. 8 illustrates the prediction value of two genes from the E2F network in the 080 trial data set.

FIG. 9 illustrates the tree-based method applied to identify additional markers to combine with ER to predict the pCR response to ixabepilone.

FIG. 10 illustrates a scatter plot of KLK6 vs. ER expression.

FIG. 11 illustrates GSEA analysis of the ER negative subpopulation. An arrow highlights a few kallikrein members and SERPINB5 and LY6D genes.

FIG. 12 illustrates a scatter plot of PR vs. ER expression.

### DETAILED DESCRIPTION OF THE INVENTION

The invention provides biomarkers that correlate with microtubule-stabilization agent sensitivity or resistance. These biomarkers can be employed for predicting response to one or more microtubule-stabilization agents. In one aspect, the biomarkers of the invention are those provided in Table 2, Table 3, and the Sequence Listing, including both polynucleotide and polypeptide sequences.

The biomarkers provided in Tables 2 and 3 include the nucleotide sequences of SEQ ID NOS:1-12 and 23-29 and the amino acid sequences of SEQ ID NOS:13-22 and 30-35.

### Microtubule-Stabilizing Agents

Agents that affect microtubule-stabilization are well known in the art. These agents have cytotoxic activity against rapidly proliferating cells, such as, tumor cells or other hyperproliferative cellular disease.

In one aspect, the microtubule-stabilizing agent is an epothilone, or analog or derivative thereof. The epothilones, including analogs and derivatives thereof, may be found to exert microtubule-stabilizing effects similar to paclitaxel (Taxol®) and, hence, cytotoxic activity against rapidly proliferating cells, such as, tumor cells or other hyperproliferative cellular disease.

Suitable microtubule-stabilizing agents are disclosed, for example, in the following PCT publications hereby incorporated by reference: WO93/10121; WO98/22461; WO99/02514; WO99/58534; WO00/39276; WO02/14323; WO02/72085; WO02/98868; WO03/070170; WO03/77903; WO03/78411; WO04/80458; WO04/56832; WO04/14919; WO03/92683; WO03/74053; WO03/57217; WO03/22844; WO03/103712; WO03/07924; WO02/74042; WO02/67941; WO01/81342; WO00/66589; WO00/58254; WO99/43320; WO99/42602; WO99/39694; WO99/16416; WO 99/07692; WO99/03848; WO99/01124; and WO 98/25929.

In another aspect, the microtubule-stabilizing agent is ixabepilone. Ixabepilone is a semi-synthetic analog of the natural product epothilone B that binds to tubulin in the same binding site as paclitaxel, but interacts with tubulin differently. (P. Giannakakou et al., P.N.A.S. USA, 97, 2904-2909 (2000)).

In another aspect, the microtubule-stabilizing agent is a taxane. The taxanes are well known in the art and include, for example, paclitaxel (Taxol®) and docetaxel (Taxotere®).

### Biomarkers and Biomarker Sets

The invention includes individual biomarkers and biomarker sets having both diagnostic and prognostic value in disease areas in which microtubule-stabilization and/or cytotoxic activity against rapidly proliferating cells, such as, tumor cells or other hyperproliferative cellular disease is of importance, e.g., in cancers or tumors. The biomarker sets comprise a plurality of biomarkers such as, for example, a plurality of the biomarkers provided in Table 2 and Table 3, that highly correlate with resistance or sensitivity to one or more microtubule-stabilizing agents.

The biomarker sets of the invention enable one to predict or reasonably foretell the likely effect of one or more microtubule-stabilizing agents in different biological systems or for cellular responses. The biomarker sets can be used in in vitro assays of microtubule-stabilizing agent response by test cells to predict in vivo outcome. In accordance with the invention, the various biomarker sets described herein, or the combination of these biomarker sets with other biomarkers or markers, can be used, for example, to predict how patients with cancer might respond to therapeutic intervention with one or more microtubule-stabilizing agents.

A biomarker set of cellular gene expression patterns correlating with sensitivity or resistance of cells following exposure of the cells to one or more microtubule-stabilizing agents provides a useful tool for screening one or more tumor samples before treatment with the microtubule-stabilizing agent. The screening allows a prediction of cells of a tumor sample exposed to one or more microtubule-stabilizing agents, based on the expression results of the biomarker set, as to whether or not the tumor, and hence a patient harboring the tumor, will or will not respond to treatment with the microtubule-stabilizing agent.

The biomarker or biomarker set can also be used as described herein for monitoring the progress of disease treatment or therapy in those patients undergoing treatment for a disease involving a microtubule-stabilizing agent.

The biomarkers also serve as targets for the development of therapies for disease treatment. Such targets may be particularly applicable to treatment of breast cancers or tumors. Indeed, because these biomarkers are differentially expressed in sensitive and resistant cells, their expression patterns are correlated with relative intrinsic sensitivity of cells to treatment with microtubule-stabilizing agents. Accordingly, the biomarkers highly expressed in resistant cells may serve as targets for the development of new therapies for the tumors which are resistant to microtubule-stabilizing agents.

The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, Taqman, or RNAse protection assay.

### Microarrays

The invention also includes specialized microarrays, e.g., oligonucleotide microarrays or cDNA microarrays, comprising one or more biomarkers, showing expression profiles that correlate with either sensitivity or resistance to one or more microtubule-stabilizing agents. Such microarrays can be employed in in vitro assays for assessing the expression level of the biomarkers in the test cells from tumor biopsies, and determining whether these test cells are likely to be resistant or sensitive to microtubule-stabilizing agents. For example, a specialized microarray can be prepared using all the biomarkers, or subsets thereof, as described herein and shown in Table 2 and Table 3. Cells from a tissue or organ biopsy can be isolated and exposed to one or more of the microtubule-stabilizing agents. Following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a resistant or a sensitive profile of gene expression. Whether or not the tested cells from a tissue or organ biopsy will respond to one or more of the microtubule-stabilizing agents and the course of treatment or therapy can then be determined or evaluated based on the information gleaned from the results of the specialized microarray analysis.

### Antibodies

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

### Kits

The invention also includes kits for determining or predicting whether a patient would be susceptible or resistant to a treatment that comprises one or more microtubule-stabilizing agents. The patient may have a cancer or tumor such as, for example, a breast cancer or tumor. Such kits would be useful in a clinical setting for use in testing a patient's biopsied tumor or other cancer samples, for example, to determine or predict if the patient's tumor or cancer will be resistant or sensitive to a given treatment or therapy with a microtubule-stabilizing agent. The kit comprises a suitable container that comprises: one or more microarrays, e.g., oligonucleotide microarrays or cDNA microarrays, that comprise those biomarkers that correlate with resistance and sensitivity to microtubule-stabilizing agents; one or more microtubule-stabilizing agents for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like, as further described herein.

### Application of Biomarkers and Biomarker Sets

The biomarkers and biomarker sets may be used in different applications. Biomarker sets can be built from any combination of biomarkers listed in Table 2 and Table 3 to make predictions about the likely effect of any microtubule-stabilizing agent in different biological systems. The various biomarkers and biomarkers sets described herein can be used, for example, as diagnostic or prognostic indicators in disease management, to predict how patients with cancer might respond to therapeutic intervention with a microtubule-stabilizing agent, and to predict how patients might respond to therapeutic intervention that affects microtubule-stabilization and/or cytotoxic activity against rapidly proliferating cells, such as, tumor cells or other hyperproliferative cellular disease.

The biomarkers have both diagnostic and prognostic value in diseases areas in which microtubule-stabilization and/or cytotoxic activity against rapidly proliferating cells, such as, tumor cells or other hyperproliferative cellular disease is of importance.

In accordance with the invention, cells from a patient tissue sample, e.g., a tumor or cancer biopsy, can be assayed to determine the expression pattern of one or more biomarkers prior to treatment with one or more microtubule-stabilizing agents. In one aspect, the tumor or cancer is breast cancer. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the microtubule-stabilizing agent, it is highly likely or predicted that the individual's cancer or tumor will respond favorably to treatment with the microtubule-stabilizing agent. By contrast, if the test cells show a biomarker expression pattern corresponding to that of the biomarkers of the control panel of cells which are resistant to the microtubule-stabilizing agent, it is highly likely or predicted that the individual's cancer or tumor will not respond to treatment with the microtubule-stabilizing agent.

The invention also provides a method of monitoring the treatment of a patient having a disease treatable by one or more microtubule-stabilizing agents. The isolated test cells from the patient's tissue sample, e.g., a tumor biopsy or tumor sample, can be assayed to determine the expression pattern of one or more biomarkers before and after exposure to a microtubule-stabilizing agent. The resulting biomarker expression profile of the test cells before and after treatment is compared with that of one or more biomarkers as described and shown herein to be highly expressed in the control panel of cells that are either resistant or sensitive to a microtubule-stabilizing agent. Thus, if a patient's response is sensitive to treatment by a microtubule-stabilizing agent, based on correlation of the expression profile of the one or biomarkers, the patient's treatment prognosis can be qualified as favorable and treatment can continue. Also, if, after treatment with a microtubule-stabilizing agent, the test cells don't show a change in the biomarker expression profile corresponding to the control panel of cells that are sensitive to the microtubule-stabilizing agent, it can serve as an indicator that the current treatment should be modified, changed, or even discontinued. This monitoring process can indicate success or failure of a patient's treatment with a microtubule-stabilizing agent and such monitoring processes can be repeated as necessary or desired.

The biomarkers of the invention can be used to predict an outcome prior to having any knowledge about a biological system. Essentially, a biomarker can be considered to be a statistical tool. Biomarkers are useful in predicting the phenotype that is used to classify the biological system.

Although the complete function of all of the biomarkers are not currently known, some of the biomarkers are likely to be directly or indirectly involved in microtubule-stabilization and/or cytotoxic activity against rapidly proliferating cells. In addition, some of the biomarkers may function in metabolic or other resistance pathways specific to the microtubule-stabilizing agents tested. Notwithstanding, knowledge about the function of the biomarkers is not a requisite for determining the accuracy of a biomarker according to the practice of the invention.

### EXAMPLES

### Example 1

### Identification of Biomarkers

CA163-080 Trial

CA163-080 (080 trial) is an exploratory genomic phase II study that was conducted in breast cancer patients who received ixabepilone as a neoadjuvant treatment. The primary objective of this study was to identify predictive markers of response to ixabepilone through gene expression profiling of pre-treatment breast cancer biopsies. Patients with invasive stage IIA-IIIB breast adenocarcinoma (tumor size ≧3 cm diameter) received 40 mg/m² ixabepilone as a 3-hour infusion on Day for up to four 21-day cycles, followed by surgery within 3-4 weeks of completion of chemotherapy. A total of 164 patients were enrolled in this study. Biopsies for gene expression analysis were obtained both pre- and post-treatment. Upon isolation of biopsies from the patients, samples were either snap frozen in liquid nitrogen or placed into RNAlater solution overnight, followed by removal from the RNAlater solution. All samples were kept at −70° C. until use.

### Evaluation of Pathological Response

Pathological response was assessed using the Sataloff classification system (D. Sataloff et al., J. Am. Coll. Surg., 180(3):297-306 (1995)) and used as an end point for the pharmacogenomic analysis. The pathologic response was evaluated in the primary tumor site at the end of treatment and prior to surgery by assessing histologic changes compared with baseline as following: At the primary tumor site, cellular modifications were evaluated in both the infiltrating tumoral component and in the possible ductal component, to determine viable residual infiltrating component (% of total tumoral mass); residual ductal component (% of total tumoral mass); the mitotic index. Pathologic Complete Response (pCR) in the breast only was defined as T-A, Total or near total therapeutic effect in primary site. Based on this criteria, responders included patients with pCR while non-responders included patients who failed to demonstrate pCR. The response rate was defined as the number of responders divided by the number of treated patients.

### Gene Expression Profiling

Total RNA was isolated using the RNeasy Mini kit (Qiagen) according to the manufacturer's instructions by Karolinska Institute (Stockholm, Sweden). A total of 134 patients with more than 1 μg of total RNA with good quality were included in the dataset for the final genomic analysis. Samples were profiled in a randomized order by batches to minimize the experimental bias. Each batch consisted of about 15 subject samples and 2 experimental controls using RNA extracted from HeLa cells. The expression profiling was done following a complete randomization with an effort to balance the number of samples from two tissue collection procedures (RNAlater and liquid nitrogen), two mRNA preparation methods (standard and DNA supernatants), tissue collection sites, and time of RNA sample preparation within in each batch. The mRNA samples from each subject was processed with HG-U133A 2.0 GeneChip® arrays on the Affymetrix platform and quantitated with GeneChip®Operating Software (GCOS) V1.0 (Affymetrix). The HG-U133A 2.0 GeneChip® array consists of about 22,276 probe sets, each containing about 15 perfect match and corresponding mismatch 25mer oligonucleotide probes from specific gene sequences.

### Gene Expression Data Processing

The gene expression data were transformed using base two logarithm. The Robust Multichip Average (RMA) method (R. Irizarry et al., Nucleic Acids Research 31(4):e15 (2003)) was used to normalize the raw expression data. The gene expression measures of each gene were centered at zero and resealed to have a 1-unit standard deviation.

Based on the definition of Pathology Complete Response (pCR), there are 23 responders and 111 non-responders in the 080 trial. Estrogen receptor 1 (ER) was previously found to be the best single-gene predictor with negative prediction value (NPV) 92% and positive prediction value (PPV) 37%, as described in PCT Publication No. ______ (PCT Application No. PCT/US2005/043261).

One consideration is that ER itself only predicts approximately 40% of the responder cases. This may be accounted for by unknown mechanisms that contribute to the resistance or sensitivity to ixabepilone treatment. In addition, there may be additional markers to combine with ER to achieve a PPV of 50% or more, while maintaining the NPV around 90%.

In this study, a pathway enrichment analysis named Gene Set Enrichment Analysis (GSEA) (A. Subramanian et al., P.N.A.S. USA., 102(43):15545-50 (2005)) was applied to the 080 trial data analysis. Based upon the pCR definition, 134 patient samples could be divided into two categories: responders (23 cases) and non-responders (111 cases). Two gene set enrichment databases (metabolic and signal pathway collection and transcription regulation cis-element collection) were tested against these two categories. Results from GSEA were further refined and confirmed by Ingenuity Pathway System and MetaCore GeneGo network system. To search for additional biomarkers that help boost PPV in combination with ER, both statistical methods and pathway analyses were used in the study. Finally, the difference of the PR expression profile between the responders versus non-responders was investigated.

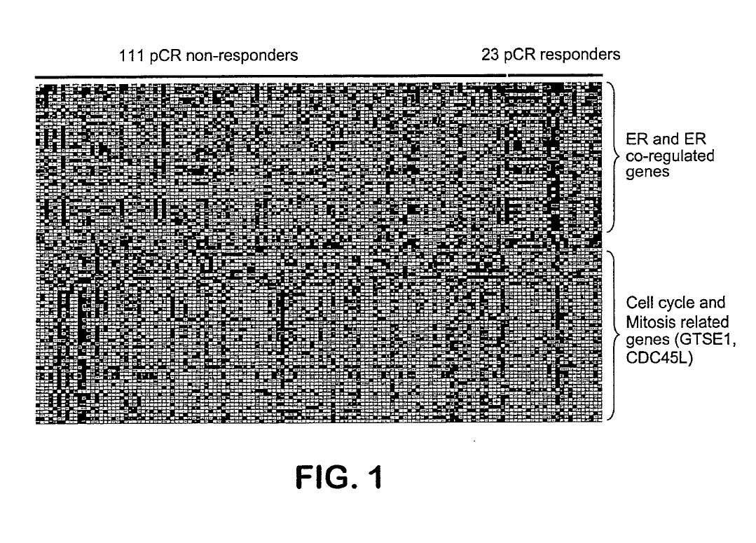

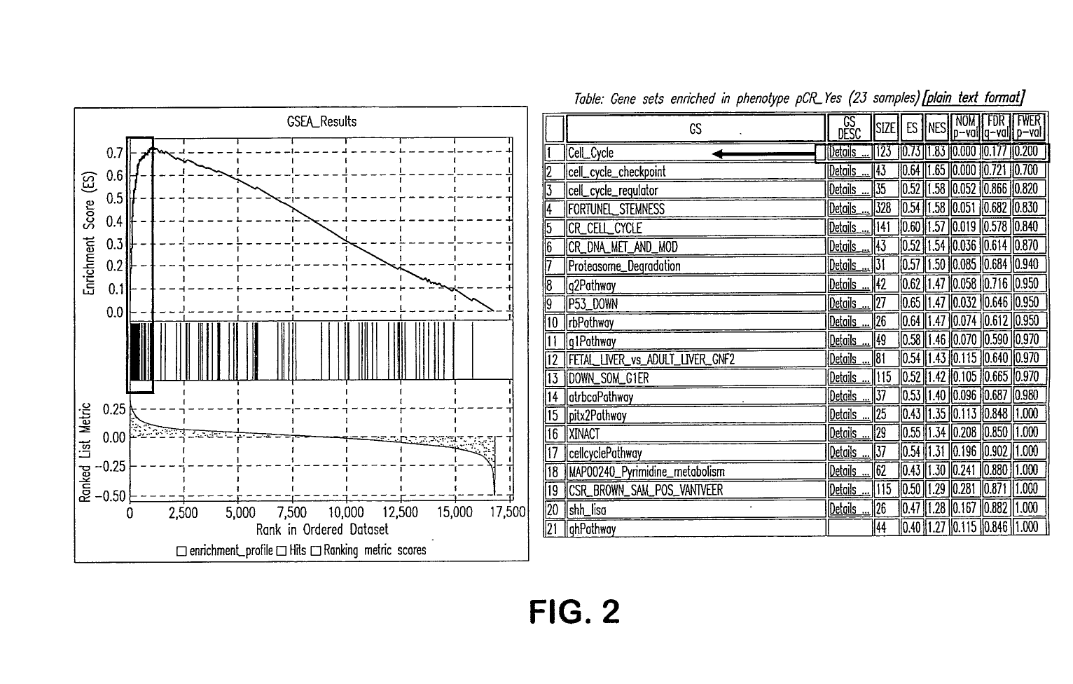

Gene Set Enrichment Analysis (GSEA) was applied to analyze the difference between responders and non-responders. FIG. 1 shows the top 100 genes that are different between these two categories. Consistent with previous observations, ER and many ER co-regulated genes are down-regulated in the responding group. Interestingly, many cell cycle related genes such as BUB1, cdc6, cdc45L, and GTSE1 are all higher at the transcription level when compared to those in the non-responding group. The most significant pathway identified by GSEA is the cell cycle pathway with p=0 and FDR q=0.177 (FIG. 2).

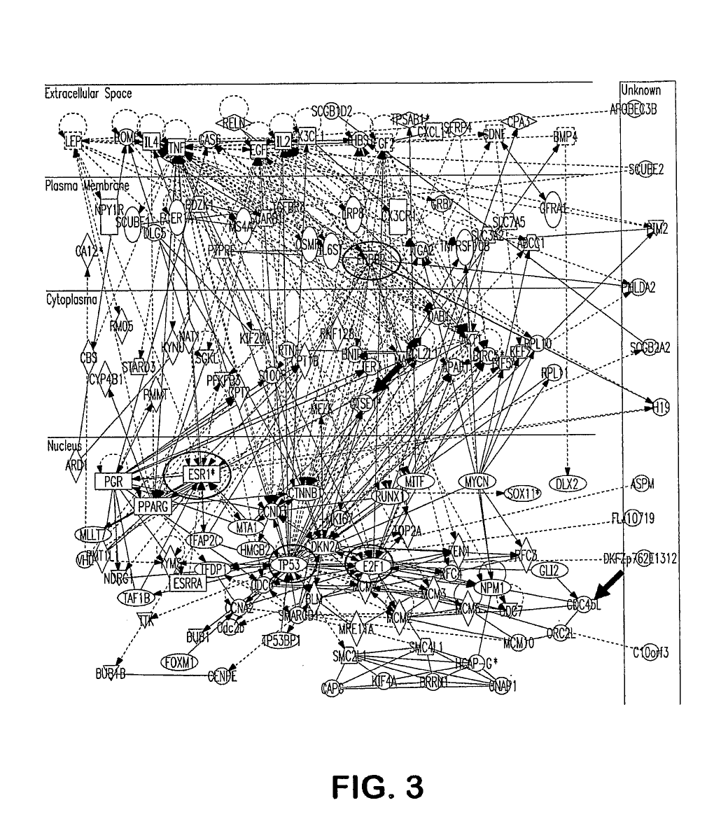

When the top 100 genes were uploaded into the Ingenuity System, the most significant gene network was shown (FIG. 3). There are at least 4 obvious pathways: ER; EGFR-ERBB2/Her2 signaling pathways; p53; and E2F transcription regulation pathways. Many genes reportedly regulated by ER were low as the ER level in the ixabepilone responding group while many genes within the p53 and E2F circuit were high.

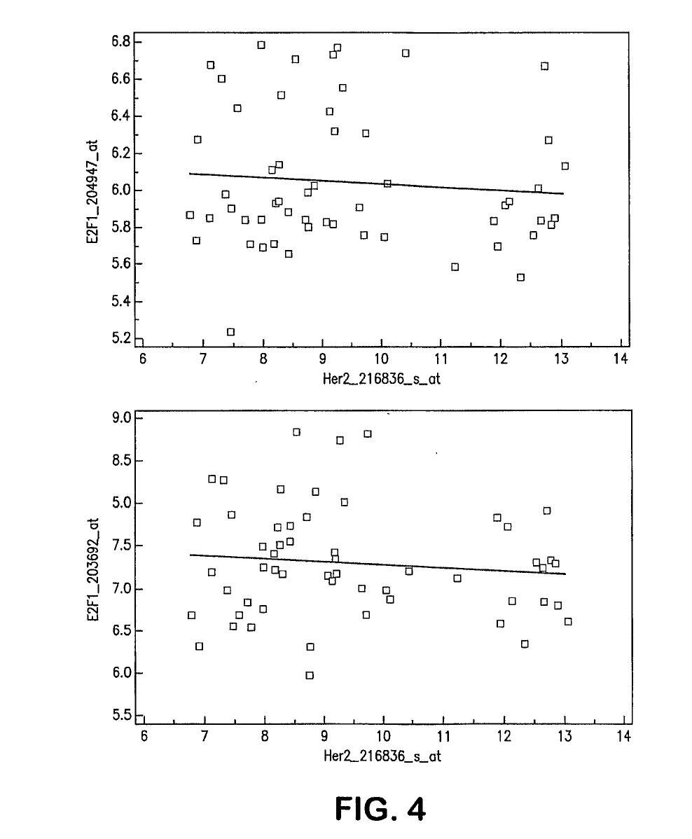

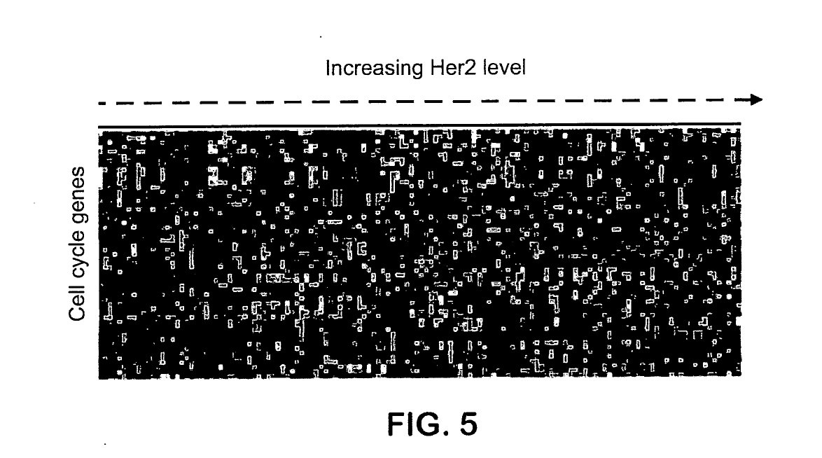

It is well known that the ER and Her2 pathways play pivotal roles in breast carcinogenesis. One hypothesis is that ER and/or Her2 may directly or indirectly affect the expression of these cell cycle related genes. To address this hypothesis, the relationship of the expression of these cell cycle related genes and the ER or Her2 level was investigated. FIG. 4 shows two scatter plots that illustrate the relationship between Her2 and E2F1 or E2F3 at the transcription level. It is clear that there is no expression correlation between them within the 080 trial data set, suggesting the Her2 level does not affect the expression of E2F1 or E2F3 gene. FIG. 5 is a heatmap showing the expression of these cell cycle genes with the increasing level of Her2 across the 134 samples. Clearly, high or lower levels of these cell cycle genes have no relationship with the level of Her2 in cancer cells.

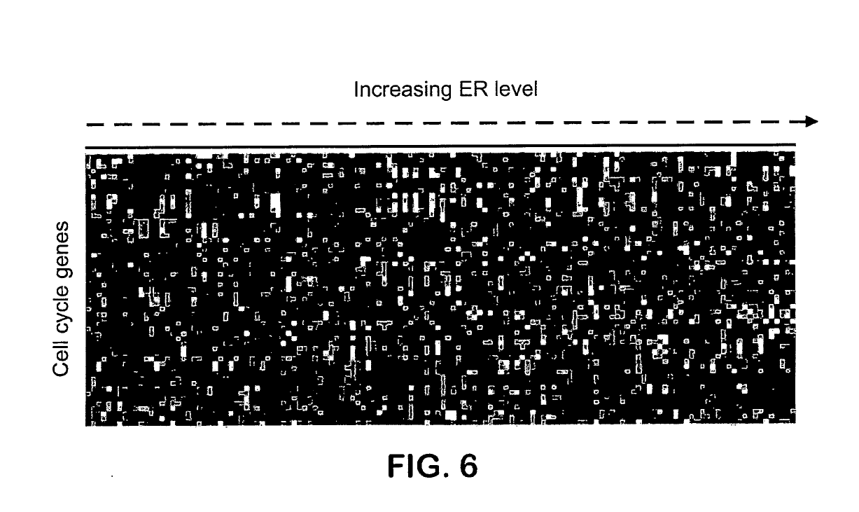

ER is known to regulate many genes in breast cancer cells. Whether or not ER also regulates these cell cycle genes is an obvious question to be addressed. Like the Her2 approach, the ER's level across the samples was sorted to compare the expression levels of these cell cycle genes among the individual samples (FIG. 6). It is clear that no correlation between the expression level of ER versus the expression levels of these cell cycle genes.

From the above results, the hypothesis set above is wrong since neither ER nor Her2 affects the expression of these cell cycle genes. The next question to address, then, is what causes the high expression in the ixabepilone responding cases versus non-responding cases. It is likely that these genes may share common regulatory machineries.

To answer this question, the cell cycle related genes were further investigated with the help of the Ingenuity System. It is obvious that this is an E2F centric gene network. All genes directly or indirectly connected to the E2F were up-regulated in the ixabepilone responding cases. The result was further confirmed by a similar gene network analysis tool called GeneGo system.

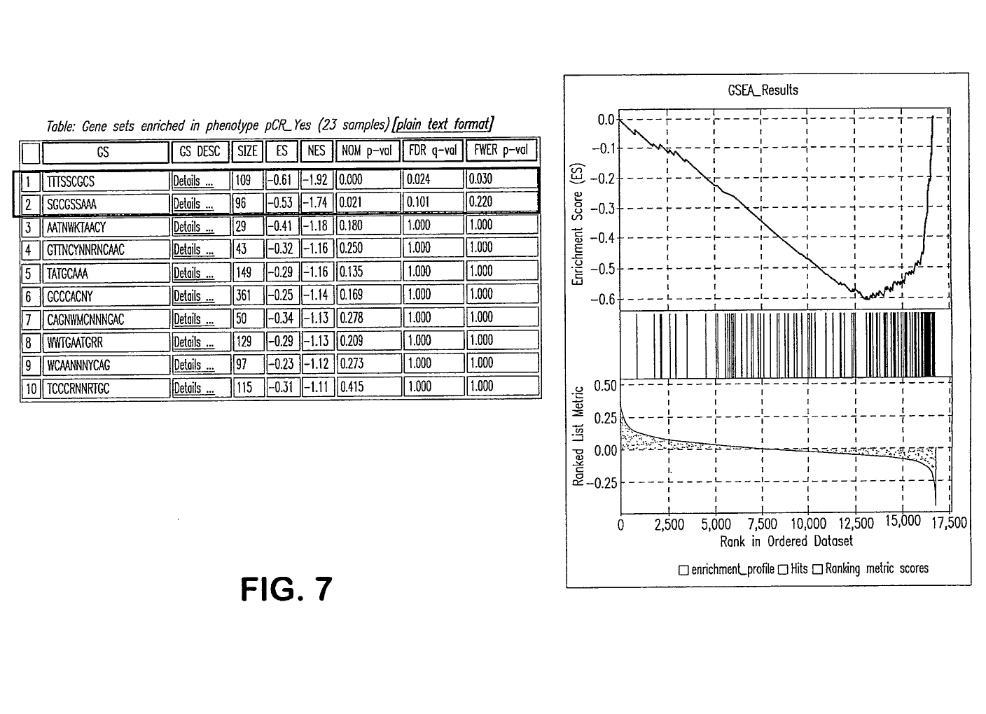

When the promoters of these cell cycle genes including E2F were examined by GSEA, it was found that they all share a common cis-element TTTSSCGCS (or SGCGSSAAA in the reverse strand). FIG. 7 shows that the calculated p value was close to zero with FDR q score around 0.02. The genes sharing these cis-elements are E2F1 and E2F3; GATA binding protein 1; interleukin enhancer binding factor, and many other cell cycle related genes.

It is interesting to know which transcription factors bind to this cis-element, and to amplify the expression of the whole set of genes in the ixabepilone responding cases. Searching the transcription factor database found that the cis-element is a binding element of E2F transcription factors.

This finding was surprising since both E2F1 and E2F3 are also on the list of those cell cycle related genes found from the ixabepilone responding group. In fact, it has been reported that E2F transcription factors have the characteristic of self amplification machinery. This means each E2F gene's promoter has its own binding element and the E2F protein could bind to its own promoter and enhance its own transcription. What causes the dis-regulation of the E2F and its gene network in some breast cancer patients is an imperative question to be addressed. The significance of this question is two-fold: (i) to seek new drug targets for breast cancer patients; and (ii) unique expression of genes within the E2F network could be pharmacogenomic biomarkers to predict patients' sensitivity to ixabepilone treatment.

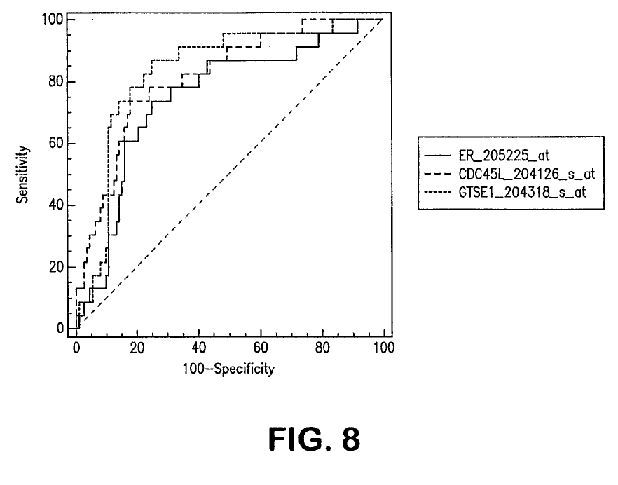

FIG. 8 demonstrates the prediction value of two genes from the E2F network in the 080 trial data set. Both CDC45L and GTSE1 (G-2 and S-phase expressed 1) are reported to play important roles in cell cycle and regulated by E2F. The following results were obtained:

ER_{—}205225_at

CDC45L_{—}204126_s_at

Difference between areas=0.067

Standard error=0.062

95% Confidence interval=−0.053 to 0.188

Significance level P=0.274

ER_{—}205225_at

GTSE1_{—}204318_s_at

Difference between areas=0.087

Standard error=0.060

95% Confidence interval=−0.032 to 0.205

Significance level P=0.152

CDC45L_{—}204126_s_at

GTSE1_{—}204318_s_at

Difference between areas=0.019

Standard error=0.049

95% Confidence interval=−0.077 to 0.115

Significance level P=0.695

From the Receiver Operating Characteristic (ROC) curve analysis, it was discovered that CDC45L and GTSE1 each have similar predictive power as ER in predicting the patients' response to ixabepilone treatment in the 080 trial.

The next question that was investigated is whether there are additional markers that can be used to combine with ER to predict pathological complete response to ixabepilone for improving the positive prediction value (PPV), negative prediction value (NPV), sensitivity, and specificity.

Tree-Based Analysis

A free-based modeling approach (Tree package in R) was applied to identify additional markers to combine with ER to predict the pCR response to ixabepilone.

In order to retain genes with good dynamic ranges so that they can be of practical use in prognostics, only genes with wide expression range in the analysis were focused on. Genes were excluded from the analysis if the difference between its maximum and minimum expression across all the 134 patient samples was less than 6. Genes were also excluded from the analysis lithe difference between its maximum and minimum expression across all the ER negative patients was less than 6. After the filtering, the resulting list only consisted of 361 genes.

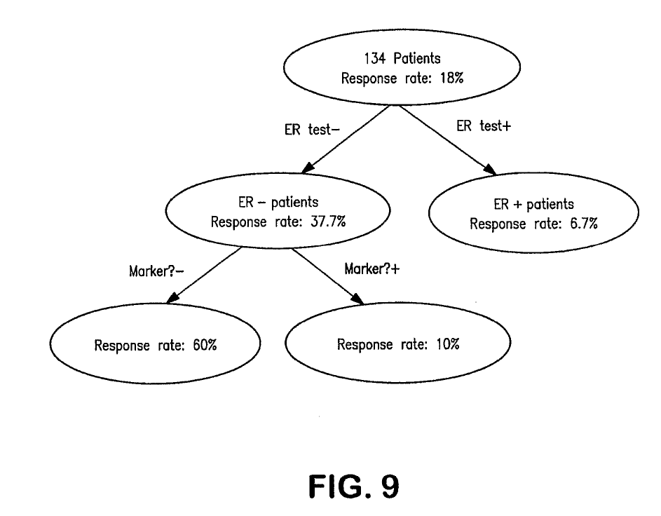

The tree-based approach proceeded as follows. At the root, the tree was split into two branches based on the ER expression. After the first split, the two child nodes were allowed to split further based on one of the 361 genes one at a time (see FIG. 9). 361 tree models, therefore, were built for the prediction purpose. Five-fold cross-validation was used to evaluate each of the tree-based models. Namely, the 134 patients were partitioned into 5 subsets randomly. The first subset was held out as the test set, and the rest of the 4 subsets were used to build the tree-based model as described above. This procedure can be repeated five times by holding different subsets as the test set. The cross-validation prediction error, PPV, NPV, sensitivity, and specificity were estimated by averaging the five prediction errors, PPV, NPV, sensitivity, and specificity, respectively, obtained on the test sets.

Table 1 presents the top 12 genes in the order of positive predictive values (PPV) in the tree-based modeling approach. Among the genes, the probe set 204773_at bad the highest sensitivity and the smallest prediction error. This gene, known as KLK6, has also been reported to be a potential biomarker for diagnosis and prognosis of Ovarian Carcinoma (E. Diamandis et al., Journal of Clinical Oncology, Vol. 21, Issue 6: 1035-1043 (2003)). Another member of the Kallikrein family, KLK10, was also found in the list

TABLE 1
PPV, NPV, Sensitivity, Specificity, and Prediction Error
of the top 12 genes in the tree-based analysis

| | | | | | Prediction |
|---|---|---|---|---|---|
| Probe_ID | PPV | NPV | Sensitivity | Specificity | error |
| 209301_at | 0.81 | 0.87 | 0.29 | 0.99 | 0.36 |
| 206276_at | 0.77 | 0.88 | 0.33 | 0.98 | 0.35 |
| 204602_at | 0.73 | 0.86 | 0.26 | 0.98 | 0.38 |
| 204733_at | 0.58 | 0.88 | 0.41 | 0.94 | 0.33 |
| 204855_at | 0.58 | 0.86 | 0.27 | 0.96 | 0.38 |
| 202917_s_at | 0.56 | 0.85 | 0.2 | 0.97 | 0.41 |
| 204846_at | 0.56 | 0.85 | 0.16 | 0.97 | 0.43 |
| 210020_x_at | 0.56 | 0.86 | 0.28 | 0.95 | 0.38 |
| 214774_x_at | 0.55 | 0.87 | 0.3 | 0.95 | 0.38 |
| 209792_s_at | 0.53 | 0.88 | 0.36 | 0.93 | 0.35 |
| 201952_at | 0.51 | 0.85 | 0.16 | 0.96 | 0.44 |
| 209800_at | 0.5 | 0.88 | 0.4 | 0.92 | 0.34 |

The 12 gene names and their affymetrix description are provided in Table 2.

TABLE 2
Top 12 genes in the tree-based analysis

| Unigene title and SEQ | | Affymetrix Probe |
|---|---|---|
| ID NO: | Affymetrix Description | Set |
| CA2: carbonic | gb:M36532.1 /DEF = Human carbonic | 209301_at |
| anhydrase II (LOC760) | anhydrase II mRNA, complete cds. | |
| SEQ ID NOS: 1 (DNA) | /FEA = mRNA /GEN = CA2 | |
| and 13 (amino acid) | /DB_XREF = gi:179794 | |
| | /UG = Hs.155097 carbonic anhydrase | |
| | II /FL = gb:J03037.1 gb:M36532.1 | |
| | gb:NM_000067.1 | |
| LY6D: lymphocyte | gb:NM_003695.1 /DEF = Homo | 206276_at |
| antigen 6 complex, locus | sapiens lymphocyte antigen 6 | |
| D (LOC8581) | complex, locus D (E48), mRNA. | |
| SEQ ID NOS: 2 (DNA) | /FEA = mRNA /GEN = E48 | |
| and 14 (amino acid) | /PROD = lymphocyte antigen 6 | |
| | complex, locus D | |
| | /DB_XREF = gi:11321574 | |
| | /UG = Hs.3185 lymphocyte antigen 6 | |
| | complex, locus D | |
| | /FL = gb:NM_003695.1 | |
| DKK1: dickkopf | gb:NM_012242.1 /DEF = Homo | 204602_at |
| homolog 1 (Xenopus | sapiens dickkopf (Xenopus laevis) | |
| laevis) (LOC22943) | homolog 1 (DKK1), mRNA. | |
| SEQ ID NOS: 3 (DNA) | /FEA = mRNA /GEN = DKK1 | |
| and 15 (amino acid) | /PROD = dickkopf (Xenopus laevis) | |
| | homolog 1 /DB_XREF = gi:7110718 | |
| | /UG = Hs.40499 dickkopf (Xenopus | |
| | laevis) homolog 1 | |
| | /FL = gb:AF127563.1 gb:AF177394.1 | |
| | gb:NM_012242.1 | |
| KLK6: kallikrein 6 | gb:NM_002774.1 /DEF = Homo | 204733_at |
| (neurosin, zyme) | sapiens kallikrein 6 (neurosin, zyme) | |
| (LOC5653) | (KLK6), mRNA. /FEA = mRNA | |
| SEQ ID NOS: 4 (DNA) | /GEN = KLK6 /PROD = kallikrein 6 | |
| and 16 (amino acid) | (neurosin, zyme) | |
| | /DB_XREF = gi:4506154 | |
| | /UG = Hs.79361 kallikrein 6 (neurosin, | |
| | zyme) /FL = gb:U62801.1 | |
| | gb:D78203.1 gb:AF013988.1 | |
| | gb:NM_002774.1 | |
| SERPINB5: serine (or | gb:NM_002639.1 /DEF = Homo | 204855_at |
| cysteine) proteinase | sapiens serine (or cysteine) proteinase | |
| inhibitor, clade B | inhibitor, clade B (ovalbumin), | |
| (ovalbumin), member 5 | member 5 (SERPINB5), mRNA. | |
| (LOC5268) | /FEA = mRNA /GEN = SERPINB5 | |
| SEQ ID NOS: 5 (DNA) | /PROD = serine (or cysteine) | |
| and 17 (amino acid) | proteinase inhibitor, cladeB | |
| | (ovalbumin), member 5 | |
| | /DB_XREF = gi:4505788 | |
| | /UG = Hs.55279 serine (or cysteine) | |
| | proteinase inhibitor, clade B | |
| | (ovalbumin), member 5 | |
| | /FL = gb:NM_002639.1 gb:U04313.1 | |
| S100A8: S100 calcium | gb:NM_002964.2 /DEF = Homo | 202917_s_at |
| binding protein A8 | sapiens S100 calcium-binding protein | |
| (calgranulin A) | A8 (calgranulin A) (S100A8), | |
| (LOC6279) | mRNA. /FEA = mRNA | |
| SEQ ID NOS: 6 (DNA) | /GEN = S100A8 /PROD = S100 | |
| and 18 (amino acid) | calcium-binding protein A8 | |
| | /DB_XREF = gi:9845519 | |
| | /UG = Hs.100000 S100 calcium- | |
| | binding protein A8 (calgranulin A) | |
| | /FL = gb:NM_002964.2 | |
| CP: ceruloplasmin | gb:NM_000096.1 /DEF = Homo | 204846_at |
| (ferroxidase) | sapiens ceruloplasmin (ferroxidase) | |
| (LOC1356) | (CP), mRNA. /FEA = mRNA | |
| SEQ ID NOS: 7 (DNA) | /GEN = CP /PROD = ceruloplasmin | |
| and 19 (amino acid) | (ferroxidase) /DB_XREF = gi:4557484 | |
| | /UG = Hs.296634 ceruloplasmin | |
| | (ferroxidase) /FL = gb:M13699.1 | |
| | gb:NM_000096.1 | |
| CALML3: calmodulin- | gb:M58026.1 /DEF = Human NB-1 | 210020_x_at |
| like 3 (LOC810) | mRNA, complete cds. /FEA = mRNA | |
| SEQ ID NOS: 8 (DNA) | /GEN = NB-1 /DB_XREF = gi:189080 | |
| and 20 (amino acid) | /UG = Hs.239600 calmodulin-like 3 | |
| | /FL = gb:M36707.1 gb:M58026.1 | |
| | gb:NM_005185.1 | |
| TNRC9: trinucleotide | Consensus includes gb:AK027006.1 | 214774_x_at |
| repeat containing 9 | /DEF = Homo sapiens cDNA: | |
| (LOC27324) | FLJ23353 fis, clone HEP14321, | |
| SEQ ID NO: 9 (DNA) | highly similar to HSU80736 Homo | |
| | sapiens CAGF9 mRNA. | |
| | /FEA = mRNA | |
| | /DB_XREF = gi:10440010 | |
| | /UG = Hs.110826 trinucleotide repeat | |
| | containing 9 | |
| KLK10: kallikrein 10 | gb:BC002710.1 /DEF = Homo | 209792_s_at |
| (LOC5655) | sapiens, kallikrein 10, clone | |
| SEQ ID NOS: 10 (DNA) | MGC:3667, mRNA, complete cds. | |
| and 21 (amino acid) | /FEA = mRNA /PROD = kallikrein 10 | |
| | /DB_XREF = gi:12803744 | |
| | /UG = Hs.69423 kallikrein 10 | |
| | /FL = gb:BC002710.1 | |
| ALCAM: activated | Consensus includes gb:AA156721 | 201952_at |
| leukocyte cell adhesion | /FEA = EST /DB_XREF = gi:1728335 | |
| molecule (LOC214) | /DB_XREF = est:zl18b04.s1 | |
| SEQ ID NO: 11 (DNA) | /CLONE = IMAGE:502255 | |
| | /UG = Hs.10247 activated leucocyte | |
| | cell adhesion molecule | |
| | /FL = gb:NM_001627.1 gb:L38608.1 | |
| KRT16: keratin 16 | gb:AF061812.1 /DEF = Homo sapiens | 209800_at |
| (focal non-epidermolytic | keratin 16 (KRT16A) mRNA, | |
| palmoplantar | complete cds. /FEA = mRNA | |
| keratoderma) | /GEN = KRT16A /PROD = keratin 16 | |
| (LOC3868) | /DB_XREF = gi:4091878 | |
| SEQ ID NOS: 12 (DNA) | /UG = Hs.115947 keratin 16 (focal | |
| and 22 (amino acid) | non-epidermolytic palmoplantar | |
| | keratoderma) /FL = gb:AF061812.1 | |
| | gb:NM_005557.1 | |

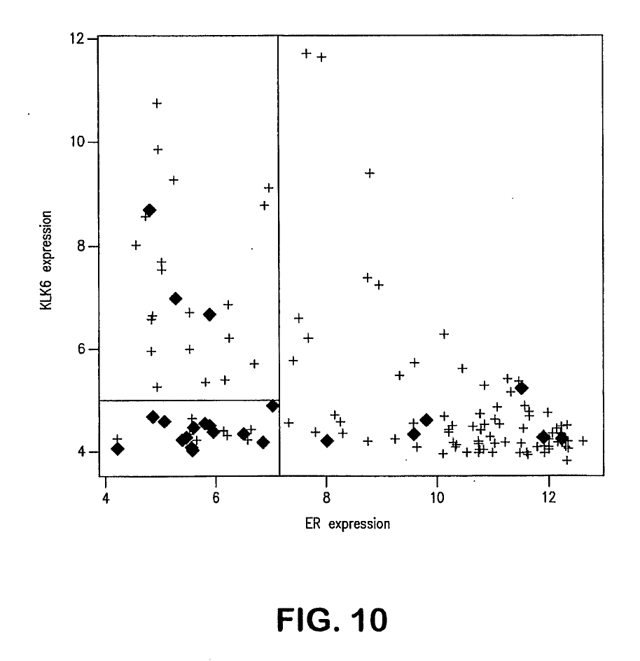

FIG. 10 is the scatter plot of the KLK6 expression and ER expression. The “+” represents the non-responders, and the dots are responders. This figure indicates that the ER expression levels of most responders were low. It was also observed that the patient is unlikely to be a responder if the ER expression was low but the KLK6 expression was high. The vertical and horizontal lines in the graph were used as the classification rule cutoff points in the tree-based model.

K-Nearest Neighbor (KNN) and GSEA Approaches

A marker gene selection process was carried out by KNN algorithm which fed only the genes with higher correlation with the target class. The KNN algorithm sets the class of the data point to the majority class appearing in the k closest training set samples. Marker filtering is done by shrinking centroids algorithm (R. Tibshirani et al., PNAS, 99(10):6567-72 (2002)) for the samples in class 1 and class 2, respectively. The euclidean distance matrix was used to determine the strength of the correlation. The magnitude of correlation values indicates the strength of the correlation between gene expression and class distinction.

In the ER-group, there were 18 responders and 37 non-responders based upon the PCR definition. A supervised learning algorithm named k-nearest neighbor (KNN) with k=3 was applied to identify potential predictors for these two categories. Table 3 shows that several kallikrein (KLK) members were on the list.

TABLE 3
Top 10 genes with the highest selection frequency by
KNN were found to distinguish the responder group versus
the non-responder group under the ER-subpopulation

| Unigene title and SEQ | | Affymetrix Probe |
|---|---|---|
| ID NO: | Affymetrix Description | Set |
| AKAP13: A kinase | gb:AF127481.1 /DEF = Homo sapiens | 209535_s_at |
| (PRKA) anchor protein | non-ocogenic Rho GTPase-specific | |
| 13 (LOC11214) | GTP exchange factor (proto-LBC) | |
| SEQ ID NOS: 23 (DNA) | mRNA, complete cds. /FEA = mRNA | |
| and 30 (amino acid) | /GEN = proto-LBC /PROD = non- | |
| | ocogenic Rho GTPase-specific GTP | |
| | exchangefactor | |
| | /DB_XREF = gi:5199315 | |
| | /UG = Hs.301946 lymphoid blast crisis | |
| | oncogene /FL = gb:AF127481.1 | |
| KLK6: kallikrein 6 | gb:NM_002774.1 /DEF = Homo | 204733_at |
| (neurosin, zyme) | sapiens kallikrein 6 (neurosin, zyme) | |
| (LOC5653) | (KLK6), mRNA. /FEA = mRNA | |
| SEQ ID NOS: 4 (DNA) | /GEN = KLK6 /PROD = kallikrein 6 | |
| and 16 (amino acid) | (neurosin, zyme) | |
| | /DB_XREF = gi:4506154 | |
| | /UG = Hs.79361 kallikrein 6 (neurosin, | |
| | zyme) /FL = gb:U62801.1 gb:D78203.1 | |
| | gb:AF013988.1 gb:NM_002774.1 | |
| SARG: specifically | gb:NM_024115.1 /DEF = Homo | 219476_at |
| androgen-regulated | sapiens hypothetical protein MGC4309 | |
| protein (LOC79098) | (MGC4309), mRNA. /FEA = mRNA | |
| SEQ ID NO: 24 (DNA) | /GEN = MGC4309 | |
| | /PROD = hypothetical protein | |
| | MGC4309 /DB_XREF = gi:13129133 | |
| | /UG = Hs.32417 hypothetical protein | |
| | MGC4309 /FL = gb:BC002325.1 | |
| | gb:BC001943.1 gb:NM_024115.1 | |
| KLK5: kallikrein 5 | Consensus includes gb:AF243527 | 222242_s_at |
| (LOC25818) | /DEF = Homo sapiens serine protease | |
| SEQ ID NOS: 25 (DNA) | gene cluster, complete sequence | |
| and 31 (amino acid) | /FEA = CDS_12 | |
| | /DB_XREF = gi:11244757 | |
| | /UG = Hs.50915 kallikrein 5 | |
| SERPINB5: serine (or | gb:NM_002639.1 /DEF = Homo | 204855_at |
| cysteine) proteinase | sapiens serine (or cysteine) proteinase | |
| inhibitor, clade B | inhibitor, clade B (ovalbumin), | |
| (ovalbumin), member 5 | member 5 (SERPINB5), mRNA. | |
| (LOC5268) | /FEA = mRNA /GEN = SERPINB5 | |
| SEQ ID NOS: 5 (DNA) | /PROD = serine (or cysteine) proteinase | |
| and 17 (amino acid) | inhibitor, cladeB (ovalbumin), member | |
| | 5 /DB_XREF = gi:4505788 | |
| | /UG = Hs.55279 serine (or cysteine) | |
| | proteinase inhibitor, clade B | |
| | (ovalbumin), member 5 | |
| | /FL = gb:NM_002639.1 gb:U04313.1 | |
| KLK7: kallikrein 7 | gb:NM_005046.1 /DEF = Homo | 205778_at |
| (chymotryptic, stratum | sapiens kallikrein 7 (chymotryptic, | |
| corneum) (LOC5650) | stratum corneum) (KLK7), mRNA. | |
| SEQ ID NOS: 26 (DNA) | /FEA = mRNA /GEN = KLK7 | |
| and 32 (amino acid) | /PROD = kallikrein 7 (chymotryptic, | |
| | stratum corneum) | |
| | /DB_XREF = gi:4826949 | |
| | /UG = Hs.15l254 kallikrein 7 | |
| | (chymotryptic, stratum corneum) | |
| | /FL = gb:NM_005046.1 gb:L33404.1 | |
| KIAA0220: PI-3- | Consensus includes gb:AI925734 | 221992_at |
| kinase-related kinase | /FEA = EST /DB_XREF = gi:5661698 | |
| SMG-1-like | /DB_XREF = est:wo34g08.xl | |
| (LOC283846) | /CLONE = IMAGE:2457278 | |
| SEQ ID NOS: 27 | /UG = Hs.110613 KIAA0220 protein | |
| (DNA) and 33 (amino | | |
| acid) | | |
| FOLR1: folate receptor | gb:NM_016725.1 /DEF = Homo | 204437_s_at |
| 1 (adult) (LOC2348) | sapiens folate receptor 1 (adult) | |
| SEQ ID NOS: 28 (DNA) | (FOLR1), transcript variant 1, mRNA. | |
| and 34 (amino acid) | /FEA = mRNA /GEN = FOLR1 | |
| | /PROD = folate receptor 1 precursor | |
| | /DB_XREF = gi:9257206 | |
| | /UG = Hs.73769 folate receptor 1 | |
| | (adult) /FL = gb:NM_000802.2 | |
| | gb:NM_016731.2 gb:BC002947.1 | |
| | gb:J05013.1 gb:NM_016725.1 | |
| | gb:NM_016729.1 | |
| LY6D: lymphocyte | gb:NM_003695.1 /DEF = Homo | 206276_at |
| antigen 6 complex, | sapiens lymphocyte antigen 6 | |
| locus D (LOC8581) | complex, locus D (E48), mRNA. | |
| SEQ ID NOS: 2 (DNA) | /FEA = mRNA /GEN = E48 | |
| and 14 (amino acid) | /PROD = lymphocyte antigen 6 | |
| | complex, locus D | |
| | /DB_XREF = gi:11321574 | |
| | /UG = Hs.3185 lymphocyte antigen 6 | |
| | complex, locus D | |
| | /FL = gb:NM_003695.1 | |
| C2orf23: chromosome 2 | Consensus includes gb:BE535746 | 204364_s_at |
| open reading frame 23 | /FEA = EST /DB_XREF = gi:9764391 | |
| (LOC65055) | /DB_XREF = est:601060419F1 | |
| SEQ ID NOS: 29 (DNA) | /CLONE = IMAGE:3446788 | |
| and 35 (amino acid) | /UG = Hs.7358 hypothetical protein | |
| | FLJ13110 /FL = gb:NM_022912.1 | |

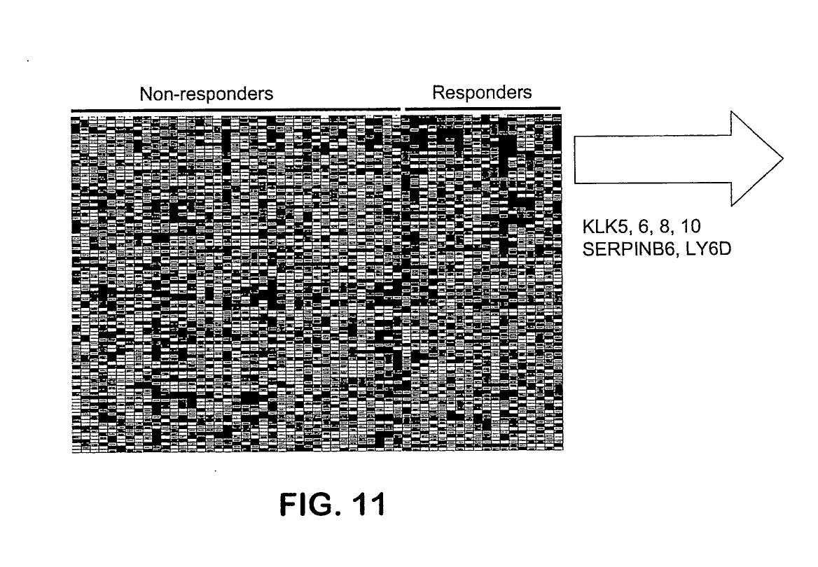

The results were further confirmed by the GSEA analysis (FIG. 11).

When the top 10 genes from the tree-based, KNN, and GSEA approaches were put together, there were 3 genes that were identified consistently by these three different methods: (i) KLK6: kallikrein 6 (neurosin, zyme) (LOC5653); (ii) SERPINB5: serine (or cysteine) proteinase inhibitor, clade 13 (ovalbumin), member 5 (LOC5268); and (iii) LY6D: lymphocyte antigen 6 complex, locus D (LOC8581).

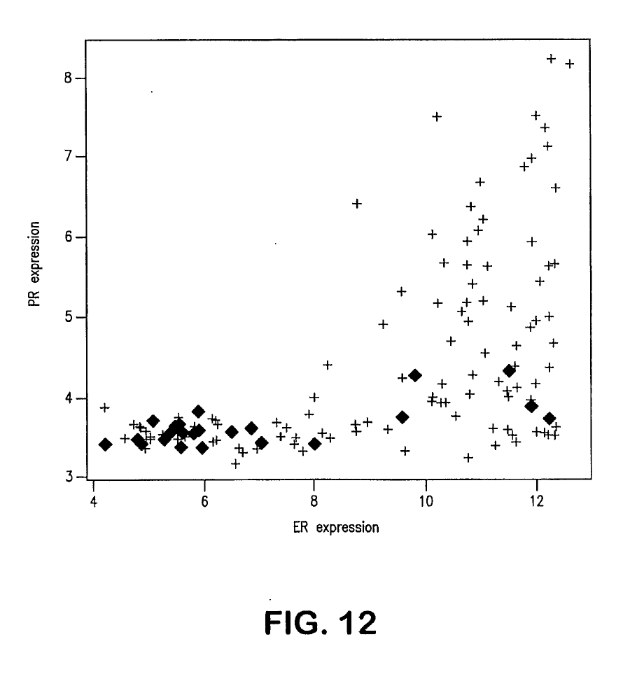

Another question to explore was the difference in the progesterone receptor (PR) gene expression between the responders and the non-responders. It was of interest to investigate the pCR in terms of the ER and PR expression. FIG. 12 shows there was some correlation (3.57) between the ER and PR expression. When the patient's ER expression is low, his PR expression was also low. It was observed in the figure that there are no patients with high PR expression but low ER expression. However, there are a number of patients whose ER expression is high but their PR expression is low.

Table 4 shows the pCR response rates for different groups by the ER and PR status. The data were based on the 080 trial. Patients' ER and PR status were determined by IHC assay. This suggests that in the ER positive group, if the PR expression level is low, the patient has a higher chance to respond to ixabepilone.

TABLE 4
Response rates by the ER and PR status

| | ER− | ER+ | |
|---|---|---|---|
| PR− | 20/61 = 33% | 4/15 = 26.7% | 24/76 = 31.5% |
| PR+ | 0/9 = 0% | 4/62 = 6% | 4/71 = 5.6% |
| | 20/70 = 29% | 8/77 = 10.4% | Overall: 18% |

Pathway enrichment analyses were applied to understand why some patients are sensitive to the ixabepilone treatment and why some are not. Up-regulation of many cell cycle genes, in particular, those whose expressions are regulated by E2F transcription factors, was found significant. High expressions of these genes are not directly or indirectly caused by ER or Her2 but believed to be regulated by E2F proteins. Two genes were found to have similar prediction values as of ER in predicting breast cancer patients' response to the ixabepilone treatment in the 080 trial based upon the AUC values.

In the effort to identify additional markers with ER to predict the pCR response rate, a list of genes was found showing certain predictability in terms of high PPV (>=0.5), NPV, sensitivity, and specificity, particularly, KLK6, SERPINB5 and LY6D were identified consistently by three methodologies as good predictors for the ixabepilone sensitivity.

In the ER positive patients, it suggested the PR status may be used to predict responders to ixabepilone.

### Example 2

### Production of Antibodies Against the Biomarkers

Antibodies against the biomarkers can be prepared by a variety of methods.

For example, cells expressing a biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1.00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC (Manassas, Va.). After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### Example 3

### Immunofluorescence Assays

The following immunofluorescence protocol may be used, for example, to verify biomarker protein expression on cells or, for example, to check for the presence of one or more antibodies that bind biomarkers expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters.

Although the invention has been described in some detail by way of illustration and example for purposes of clarity and understanding, it will be apparent that certain changes and modifications may be practiced within the scope of the appended claims.

What is claimed is:

1. A method for predicting whether a mammal will respond therapeutically to a method of treating cancer comprising administering a microtubule-stabilizing agent, wherein the method comprises:
(a) measuring in the mammal the level of at least one biomarker selected from the biomarkers of Table 2 and Table 3;
(b) exposing a biological sample from said mammal to said agent;
(c) following the exposing of step (b), measuring in said biological sample the level of the at least one biomarker,
wherein a difference in the level of the at least one biomarker measured in step (c) compared to the level of the at least one biomarker measured in step (a) indicates that the mammal will respond therapeutically to said method of treating cancer.

2. The method of claim 1 wherein said agent is an epothilone or analog or derivative thereof.

3. The method of claim 1 wherein said agent is ixabepilone.

4. The method of claim 1 wherein said agent is a taxane.

5. A method for identifying a mammal that will respond therapeutically to a method of treating cancer comprising administering a microtubule-stabilizing agent, wherein the method comprises:
(a) exposing a biological sample from the mammal to said agent;
(b) following the exposing of step (a), measuring in said biological sample the level of the at least one biomarker selected from the biomarkers of Table 2 and Table 3,
wherein a difference in the level of the at least one biomarker measured in step (b), compared to the level of the at least one biomarker in a mammal that has not been exposed to said agent, indicates that the mammal will respond therapeutically to said method of treating cancer.

6. The method of claim 5 wherein said agent is an epothilone or analog or derivative thereof.

7. The method of claim 5 wherein said agent is ixabepilone.

8. The method of claim 5 wherein said agent is a taxane.


SEQUENCE LISTING (35 sequences)

SEQ ID NO: 1 - DNA, 1551 bp, Homo sapiens
ggcgcccaagccgccgccgccagatcggtgccgattcctgccctgccccgaccgccagcgcgaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccctggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattct

SEQ ID NO: 2 - DNA, 1445 bp, Homo sapiens
cgagtgtcagatttacaatgtttcagaatctgaggatcttagaactgactcctggagtcacggaccctgagagagtggagcagggggttgtcttgagatgcttctcactgggagagcggccatctagtccctgccaccagcccctcccggtccccaggtctcccccagcacagcccaggacagcccaaaggccaccaagcccttcctgtcataggctcagagcagccaagctggctgcccccggggccttagctccgtcaggccctcagaaacatagccatctcctccagggacatcccctggggtggggacaagagccagtgtcctcttggcagtcgcagagctccctgagaccttggaggaaaggcttgcaacacaccctagacaggggcagggcaggacacctcttccaagccggcccaggctggaatacatgtcccctgaggctgcactcagggcacatgccctgccaggacccagagcccgggtttgggggcctagggcaagggcagcagggcctctgtccaggaggggtctctgcaggtgggggagcaggccctgctaagtcaccaggaggcctctgttgggcccgctgagccggactccctccccgccagcccttaccctgcgctgccacgtgtgcaccagctccagcaactgcaagcattctgtggtctgcccggccagctctcgcttctgcaagaccacgaacacaggtgggtagcatctgtccaggtgtccctggggctgggggacatccctggctgttgtgggtgttggctggaaggttgtggggaggtggggggcagaggtggctgcctggcctgaccacactcacctgtgcccagtggagcctctgagggggaatctggtgaagaaggactgtgcggagtcgtgcacacccagctacaccctgcaaggccaggtcagcagcggcaccagctccacccagtgctgccaggaggacctgtgcaatgagaagctgcacaacgctgcacccacccgcaccgccctcgcccacagtgccctcagcctggggctggccctgagcctcctggccgtcatcttagcccccagcctgtgaccttccccccagggaaggcccctcatgcctttccttccctttctctggggattccacacctctcttccccagccgcaacgggggtgccaggagccccaggctgagggcttccccgaaagtctgggaccaggtccaggtgggcatggaatgctgatgacttggagcaggccccacagaccccacagaggatgaagccaccccacagaggatgcagcccccagctgcatggaaggtggaggacagaagccctgtggatccccggatttcacactccttctgttttgttgccgtttatttttgtactcaaatctctacatggagataaatgatttaaaccagaaaaaaaaaaaaaaaaaaaaaa

SEQ ID NO: 3 - DNA, 1815 bp, Homo sapiens
gcagagctctgtgctccctgcagtcaggactctgggaccgcagggggctcccggaccctgactctgcagccgaaccggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacatacattttgggattgtgatcttagaggtttgtgtgtgtgtacgtatgtgtgtgttctacaagaacggaagtgtgatatgtttaaagatgatcagagaaaagacagtgtctaaatataagacaatattgatcagctctagaataactttaaagaaagacgtgttctgcattgataaactcaaatgatcatggcagaatgagagtgaatcttacattactactttcaaaaatagtttccaataaattaataatacctaaaaaaaaaaa

SEQ ID NO: 4 - DNA, 1527 bp, Homo sapiens
ggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaa

SEQ ID NO: 5 - DNA, 2558 bp, Homo sapiens
ttgtgctcctcgcttgcctgttccttttccacgcattttccaggataactgtgactccaggcccgcaatggatgccctgcaactagcaaattcggcttttgccgttgatctgttcaaacaactatgtgaaaaggagccactgggcaatgtcctcttctctccaatctgtctctccacctctctgtcacttgctcaagtgggtgctaaaggtgacactgcaaatgaaattggacaggttcttcattttgaaaatgtcaaagatataccctttggatttcaaacagtaacatcggatgtaaacaaacttagttccttttactcactgaaactaatcaagcggctctacgtagacaaatctctgaatctttctacagagttcatcagctctacgaagagaccctatgcaaaggaattggaaactgttgacttcaaagataaattggaagaaacgaaaggtcagatcaacaactcaattaaggatctcacagatggccactttgagaacattttagctgacaacagtgtgaacgaccagaccaaaatccttgtggttaatgctgcctactttgttggcaagtggatgaagaaatttcctgaatcagaaacaaaagaatgtcctttcagactcaacaagacagacaccaaaccagtgcagatgatgaacatggaggccacgttctgtatgggaaacattgacagtatcaattgtaagatcatagagcttccttttcaaaataagcatctcagcatgttcatcctactacccaaggatgtggaggatgagtccacaggcttggagaagattgaaaaacaactcaactcagagtcactgtcacagtggactaatcccagcaccatggccaatgccaaggtcaaactctccattccaaaatttaaggtggaaaagatgattgatcccaaggcttgtctggaaaatctagggctgaaacatatcttcagtgaagacacatctgatttctctggaatgtcagagaccaagggagtggccctatcaaatgttatccacaaagtgtgcttagaaataactgaagatggtggggattccatagaggtgccaggagcacggatcctgcagcacaaggatgaattgaatgctgaccatccctttatttacatcatcaggcacaacaaaactcgaaacatcattttctttggcaaattctgttctccttaagtggcatagcccatgttaagtcctccctgacttttctgtggatgccgatttctgtaaactctgcatccagagattcattttctagatacaataaattgctaatgttgctggatcaggaagccgccagtacttgtcatatgtagccttcacacagatagacctttttttttttccaattctatcttttgtttccttttttcccataagacaatgacatacgcttttaatgaaaaggaatcacgttagaggaaaaatatttattcattatttgtcaaattgtccggggtagttggcagaaatacagtcttccacaaagaaaattcctataaggaagatttggaagctcttcttcccagcactatgctttccttctttgggatagagaatgttccagacattctcgcttccctgaaagactgaagaaagtgtagtgcatgggacccacgaaactgccctggctccagtgaaacttgggcacatgctcaggctactataggtccagaagtccttatgttaagccctggcaggcaggtgtttattaaaattctgaattttggggattttcaaaagataatattttacatacactgtatgttatagaacttcatggatcagatctggggcagcaacctataaatcaacaccttaatatgctgcaacaaaatgtagaatattcagacaaaatggatacataaagactaagtagcccataaggggtcaaaatttgctgccaaatgcgtatgccaccaacttacaaaaacacttcgttcgcagagcttttcagattgtggaatgttggataaggaattatagacctctagtagctgaaatgcaagaccccaagaggaagttcagatcttaatataaattcactttcatttttgatagctgtcccatctggtcatgtggttggcactagactggtggcaggggcttctagctgactcgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttgggtggataagctatccctgttgccggttcatggattacttctctataaaaaatatatatttaccaaaaaattttgtgacattccttctcccatctcttccttgacatgcattgtaaataggttcttcttgttctgagattcaatattgaatttctcctatgctattgacaataaaatattattgaactacc

SEQ ID NO: 6 - DNA, 428 bp, Homo sapiens
atgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaaaaaaaaaaaaa

SEQ ID NO: 7 - DNA, 3321 bp, Homo sapiens
atgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaagctggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagagactggagcat

SEQ ID NO: 8 - DNA, 1302 bp, Homo sapiens
gagacagcccgccggccgcccggatctccacctgccaccccagagctgggacagcagccgggctgcggcactgggagggagaccccacagtggcctcttctgccacccacgcccccacccctggcatggccgaccagctgactgaggagcaggtcacagaattcaaggaggccttctccctgtttgacaaggatggggacggctgcatcaccacccgcgagctgggcacggtcatgcggtccctgggccagaaccccacggaggccgagctgcgggacatgatgagtgagatcgaccgggacggcaacggcaccgtggacttccccgagttcctgggcatgatggccaggaagatgaaggacacggacaacgaggaggagatccgcgaggccttccgcgtgttcgacaaggacggcaacggcttcgtcagcgccgccgagctgcgacacgtcatgacccggctgggggagaagctgagtgacgaggaggtggacgagatgatccgggccgcggacacggacggagacggacaggtgaactacgaggagtttgtccgtgtgctggtgtccaagtgaggccggcgcccaccatgctcctgggcgcccacgcggcccacagggcaagaacccggggcctcccgcctcctcccccatccccctgcctcccctgggcactgtggcttcctcctgcgcctggttgattcagcccacctctctgcatcccgcttcccgcgtctcttctctgcactcctgccgaccttcccacctgctcatctgaatgacacggaacgctcccactgcaggcaaaccgtgacgccctccccactcgggagaagcagagctgaccttaggaccgagcaccagggcaggttgcgctgactctgcggccctccaggacggacaccgggtgaccccttagggcacccaggcaagatccctaagaggcacccaatgcccaggccaggggggctgcagccctcagcccccgccaggattcccgcaggctcctggactggaagctccctccgcggtcggattctggagggtgggaggcatcttggcctgcagtaagcggtgctgacggggactctggccacagaggtcaggcctcctgaaaacagcactgccttccgcgctgccccagcttgccccattccttgtccgccaacccaccgtgattcatcttctgaagctgggagtgaaactgggtcagctgtaacctgttcctattcatctggaaggagggaggcttggatgagcaggggatgagagctgcagggaaataaatgagatattcgtcctt

SEQ ID NO: 9 - DNA, 3415 bp, Homo sapiens
tttgatgataatcttttcattttaaataatgtggaattttttcccctttgttcaaaaagggcactacaaagggattaggtccatcctgcttgcctttccattgatcatctttctgaacccttgtgctgtcctggtgggtttccaaactttgaagctgcctctgcgtgtctcattattgtcaccgggcatttgaaattttattccaagttcattttaaagcaaggcagtatgagaggtattcagaaattagccatgcttgtgggagatacttccttaggggcttcgtctcctctccttccccttgcaactcctccacacaaatgaagggaagaatgggttccttgggggtcgatcccccccttactccttgcatgaaactccaattaccattaaaagaagttaacttttacatatccacagaagaatagatcaactcctatgagtgataaaatagaataattgttttcaagaatgaagagtggaaagtccttgattttgtttacaaatcatgaacttatttcaggaactcattcctagaagcttgttttgttgggaaggtgcaacattagaaatttcttgttagtttgcactgagtttattactgtagatagcaggctgattttctgtcccagaaatataattctgagggactggaaaagtgctgcacctaacttttaaactctggagagaaagatgcatgttgactgaacacccagccatcagaccacttaggaaaaggagaagcccaaatcatgtttaatatgctggaattttatttatgtaactcagtggggtcactaatggaaatctcctagcttggatgatttctgactcttaattttgctccaaaagaaaaccaacattgaaatgctttgctcttttctcttctgtcttcatcattttacctgttgtttttatcctcttgtttggtatagtttagaaaggtatctgagggaaagaagacattgatggagaaactcattattttctctaaagtaagccatttgcagcaaagcttattcttcttgctaccttatttattgccttagcatgaggttttcagctgttctcaagaaaaagtgattctcaagtaatctgtattgcatccagaaatctttttcttatttgcaatagtaaatataaaaattgtgctccagtgagggtggcagggttctgttttgttttaatatccgtgtgtgtgtgtgtgtgtgtgtgtgtgtgtgtgtgtgcatgcgctaacatttaaaaatcaaagccaaggagaccaacctttttatgtgagttgtgtttatgtgacctaatttgtgcagcagctattttatagctgagaatttatacgctaaaggtggtgctaattgtggtcttttaataggtatataaaaggaaaacagaagctgccaaaaaagaatacctgaaggccctggcggcatacagggccagcctcgtttctaaggctgctgctgagtcagcagaagcccagaccatccgttctgttcagcagaccctggcgtcgaccaatctaacatcctctctccttctcaacactccactgtctcaacatggaacagtgtcagcatcacctcagactctccagcaatccctccctaggtcaatcgctcccaaacccttaaccatgagactccccatgaaccagattgtcacatcagtcaccattgcagccaacatgccctcgaacattggggctccactgataagctccatgggaacgaccatggttggctcagcaccctccacccaagtgagtccttcggtgcaaacccagcagcatcagatgcaattgcagcagcagcagcagcagcaacaacaacagatgcaacagatgcagcagcagcaactccagcagcaccaaatgcatcagcaaatccagcagcagatgcagcagcagcatttccagcaccacatgcagcagcacctgcagcagcagcagcagcatctccagcagcaaattaatcaacagcagctgcagcagcagctgcagcagcgcctccagctgcagcagctgcaacacatgcagcaccagtctcagccttctcctcggcagcactcccctgtcgcctctcagataacatcccccatccctgccatcgggagcccccagccagcctctcagcagcaccagtcgcaaatacagtctcagacacagactcaagtattatcgcaggtcagtattttctgaagacgcatatggcagacggatttgcgtataccaaggagagtggcataggagggaaaagcatatgtggctgaaacctgtaagttggtgttggttatgcagaaatgtgtaacagatcaaacggtcctctcaagtgtctattagataggcaataagaactgcagtgtagctgagtaacatcttttagctgactataaatcactttgtttttaaacaagaaaagctgtgctcttttatgtgatgccttttttatttattcaggctatacctacaatatgtgaatcaaactgtttaatgaatcctgggacatactgatgactataaactggcctctctgagtcatagaaaaatggccttatttctccagaagtgagtaaaccacacttccaggctatctgaactcctgaagccctaaaaataaaaagcacagttgtaactacctgaaatatgaagatccagtttcatacaaacatttgtatgacgtgaatagttgatggcattttttgtcatgaaaaaaataatgtaaatcacagacttttgccaaagctcttattttttttcctaaatctctccagaaaaaaaatgcaagtgactaaattcaattattgactaatttccactttttatccatgacttctccaaatcaaaccacagtatatgttgtaacaatatctatgaccactgttagcccattatattcattccaattagaagaaatgtgaatactatattccgtgttttgagtgacaagtttcgaaaaataaaaacactgtatttttaaaagggaaatgcacttaaatgaaaacagttattacaaaagttaagatttaaaaagaaaaagcaagagtttttattatgatgtaataccagtagaatatttaaaaggcacaccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctatagaataaacacatttcagtaaaaactgtggactgtattttgatgcaacaacaaagaaactgttcacttttcaaataaaatgatatgtcagatttc

SEQ ID NO: 10 - DNA, 1580 bp, Homo sapiens
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 11 - DNA, 4716 bp, Homo sapiens
cggacgcgtgggccgctgccgccgctgccgctgctaccaccgctgccacctgaggagacccgccgcccccccgtcgccgcctcctgcgagtccttcttagcacctggcgtttcatgcacattgccactgccattattattatcattccaatacaaggaaaataaaagaagataccagcgaaaagaaccgcttacacctttccgaattactcaagtgtctcctggaaacagagggtcgttgtccccggaggagcagccgaagggcccgtgggctggtgttgaccgggagggaggaggagttgggggcattgcgtggtggaaagttgcgtgcggcagagaaccgaaggtgcagcgccacagcccaggggacggtgtgtctgggagaagacgctgcccctgcgtcgggacccgccagcgcgcgggcaccgcggggcccgggacgacgccccctcctgcggcgtggactccgtcagtggcccaccaagaaggaggaggaatatggaatccaagggggccagttcctgccgtctgctcttctgcctcttgatctccgccaccgtcttcaggccaggccttggatggtatactgtaaattcagcatatggagataccattatcataccttgccgacttgacgtacctcagaatctcatgtttggcaaatggaaatatgaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacaagacaaccaaggctgacatacaaatgccattcacctgctcggtgacatattatggaccatctggccagaaaacaattcattctgaacaggcagtatttgatatttactatcctacagagcaggtgacaatacaagtgctgccaccaaaaaatgccatcaaagaaggggataacatcactcttaaatgcttagggaatggcaaccctcccccagaggaatttttgttttacttaccaggacagcccgaaggaataagaagctcaaatacttacacactaacggatgtgaggcgcaatgcaacaggagactacaagtgttccctgatagacaaaaaaagcatgattgcttcaacagccatcacagttcactatttggatttgtccttaaacccaagtggagaagtgactagacagattggtgatgccctacccgtgtcatgcacaatatctgctagcaggaatgcaactgtggtatggatgaaagataacatcaggcttcgatctagcccgtcattttctagtcttcattatcaggatgctggaaactatgtctgcgaaactgctctgcaggaggttgaaggactaaagaaaagagagtcattgactctcattgtagaaggcaaacctcaaataaaaatgacaaagaaaactgatcccagtggactatctaaaacaataatctgccatgtggaaggttttccaaagccagccattcaatggacaattactggcagtggaagcgtcataaaccaaacagaggaatctccttatattaatggcaggtattatagtaaaattatcatttcccctgaagagaatgttacattaacttgcacagcagaaaaccaactggagagaacagtaaactccttgaatgtctctgctataagtattccagaacacgatgaggcagacgagataagtgatgaaaacagagaaaaggtgaatgaccaggcaaaactaattgtgggaatcgttgttggtctcctccttgctgcccttgttgctggtgtcgtctactggctgtacatgaagaagtcaaagactgcatcaaaacatgtaaacaaggacctcggtaatatggaagaaaacaaaaagttagaagaaaacaatcacaaaactgaagcctaagagagaaactgtcctagttgtccagagataaaaatcatatagaccaattgaagcatgaacgtggattgtatttaagacataaacaaagacattgacagcaattcatggttcaagtattaagcagttcattctaccaagctgtcacaggttttcagagaattatctcaagtaaaacaaatgaaatttaattacaaacaataagaacaagttttggcagccatgataataggtcatatgttgtgtttggttcaattttttttccgtaaatgtctgcactgaggatttctttttggtttgccttttatgtaaattttttacgtagctatttttatacactgtaagctttgttctgggagttgctgttaatctgatgtataatgtaatgtttttatttcaattgtttatatggataatctgagcaggtacatttctgattctgattgctatcagcaatgccccaaactttctcataagcacctaaaacccaaaggtggcagcttgtgaagattggggacactcatattgccctaattaaaaactgtgatttttatcacaagggaggggaggccgagagtcagactgatagacaccataggagccgactctttgatatgccaccagcgaactctcagaaataaatcacagatgcatatagacacacatacataatggtactcccaaactgacaattttacctattctgaaaaagacataaaacagaatttggtagcacttacctctacagacacctgctaataaattattttctgtcaaaagaaaaaacacaagcatgtgtgagagacagtttggaaaaatcatggtcaacattcccattttcatagatcacaatgtaaatcactataattacaaattggtgttaaatcctttgggttatccactgccttaaaattatacctatttcatgtttaaaaagatatcaatcagaattggagtttttaacagtggtcattatcaaagctgtgttattttccacagaatatagaatatatattttttcgtgtgtgtttttgttaactaccctacagatattgaatgcaccttgagataatttagtgtttttaactgatacataatttatcaagcagtacatgaaagtgtaataataaaatgtctatgtatctttagttacattcaaatttgtaactttataaacatgttttatgcttgaggaaatttttaaggtggtagtataaatggaaactttttgaagtagaccagatatgggctacttgtgactagacttttaaactttgctctttcaagcagaagcctggtttctgggagaacactgcacagcgatttctttcccaggatttacacaactttaaagggaagataaatgaacatcagatttctaggtatagaactatgttattgaaaggaaaaggaaaactggtgtttgtttcttagactcatgaaataaaaaattatgaaggcaatgaaaaataaattgaaaattaaagtcagatgagaataggaataatactttgccacttctgcattatttagaaacatacgttattgtacatttgtaaaccatttactgtctgggcaatagtgactccgtttaataaaagcttccgtagtgcattggtatggattaaatgcataaaatattcttagactcgatgctgtataaaatattatgggaaaaaaagaaaatatgttattttgcctctaaacttttattgaagttttatttggcaggaaaaaaaattgaatcttggtcaacatttaaaccaaagtaaaaggggaaaaaccaaagttatttgttttgcatggctaagccattctgttatctctgtaaatactgtgatttcttttttattttctctttagaattttgttaaagaaattctaaaatttttaaacacctgctctccacaataaatcacaaacactaaaataaaattacttccatataaatattattttctcttttggtgtgggagatcaaaggtttaaagtctaacttctaagatatatttgcagaaagaagcaacatgacaatagagagagttatgctacaattatttcttggtttccacttgcaatggttaattaagtccaaaaacagctgtcagaacctcgagagcagaacatgagaaactcagagctctggaccgaaagcagaaagtttgccgggaaaaaaaaagacaacattattaccatcgattcagtgcctggataaagaggaaagcttacttgtttaatggcagccacatgcacgaagatgctaagaagaaaaagaattccaaatcctcaacttttgaggtttcggctctccaatttaactctttggcaacaggaaacaggttttgcaagttcaaggttcactccctatatgtgattataggaattgtttgtggaaatggattaacatacccgtctatgcctaaaagataataaaactgaaatatgtcttcaaaaaaaaaaaaaaaaaaaa

SEQ ID NO: 12 - DNA, 1688 bp, Homo sapiens
acagcacgctctcagccttcctgagcacctttccttctttcagccaactgctcactcgctcacctccctccttggcaccatgaccacctgcagccgccagttcacctcctccagctccatgaagggctcctgcggcatcggaggcggcatcgggggcggctccagccgcatctcctccgtcctggccggagggtcctgccgtgcccccagcacctacgggggcggcctgtctgtctcctctcgcttctcctctgggggagcctgcgggctggggggcggctatggcggtggcttcagcagcagcagcagctttggtagtggcttcgggggaggatatggtggtggccttggtgctggcttcggtggtggcttgggtgctggctttggtggtggttttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcccagtgagatcaaagactacagtccctacttcaagaccatcgaggacctgaggaacaagatcattgcggccaccattgagaatgcgcagcccattttgcagattgacaatgccaggctggcagccgatgacttcaggaccaagtatgagcacgaactggccctgcggcagactgtggaggccgacgtcaatggcctgcgccgggtgttggatgagctgaccctggccaggactgacctggagatgcagatcgaaggcctgaaggaggagctggcctacctgaggaagaaccacgaggaggagatgcttgctctgagaggtcagaccggcggagatgtgaacgtggagatggatgctgcacctggcgtggacctgagccgcatcctgaatgagatgcgtgaccagtacgagcagatggcagagaaaaaccgcagagacgctgagacctggttcctgagcaagaccgaggagctgaacaaagaagtggcctccaacagcgaactggtacagagcagccgcagtgaggtgacggagctccggagggtgctccagggcctggagattgagctgcagtcccagctcagcatgaaagcatccctggagaacagcctggaggagaccaaaggccgctactgcatgcagctgtcccagatccagggactgattggcagtgtggaggagcagctggcccagctacgctgtgagatggagcagcagagccaggagtaccagatcttgctggatgtgaagacgcggctggagcaggagattgccacctaccgccgcctgctggagggcgaggatgcccacctttcctcccagcaagcatctggccaatcctattcttcccgcgaggtcttcacctcctcctcgtcctcttcgagccgtcagacccggcccatcctcaaggagcagagctcatccagcttcagccagggccagagctcctagaactgagctgcctctaccacagcctcctgcccaccagctggcctcacctcctgaaggcccgggtcaggaccctgctctcctggcgcagttcccagctatctcccctgctcctctgctggtggtgggctaataaagctgactttctggttgatgcaaaaaaaaaaaaaaaaaaaaaaaaaaaaaaaaa

SEQ ID NO: 13 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 14 - protein, 22 aa, Homo sapiens
ECQIYNVSESEDLRTDSWSHGP

SEQ ID NO: 15 - protein, 266 aa, Homo sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 16 - protein, 244 aa, Homo sapiens
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 17 - protein, 375 aa, Homo sapiens
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 18 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 19 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 20 - protein, 149 aa, Homo sapiens
MADQLTEEQVTEFKEAFSLFDKDGDGCITTRELGTVMRSLGQNPTEAELRDMMSEIDRDGNGTVDFPEFLGMMARKMKDTDNEEEIREAFRVFDKDGNGFVSAAELRHVMTRLGEKLSDEEVDEMIRAADTDGDGQVNYEEFVRVLVSK

SEQ ID NO: 21 - protein, 276 aa, Homo sapiens
MRAPHLHLSAASGARALAKLLPLLMAQLWAAEAALLPQNDTRLDPEAYGSPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVLGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 22 - protein, 473 aa, Homo sapiens
MTTCSRQFTSSSSMKGSCGIGGGIGGGSSRISSVLAGGSCRAPSTYGGGLSVSSRFSSGGACGLGGGYGGGFSSSSSFGSGFGGGYGGGLGAGFGGGLGAGFGGGFAGGDGLLVGSEKVTMQNLNDRLASYLDKVRALEEANADLEVKIRDWYQRQRPSEIKDYSPYFKTIEDLRNKIIAATIENAQPILQIDNARLAADDFRTKYEHELALRQTVEADVNGLRRVLDELTLARTDLEMQIEGLKEELAYLRKNHEEEMLALRGQTGGDVNVEMDAAPGVDLSRILNEMRDQYEQMAEKNRRDAETWFLSKTEELNKEVASNSELVQSSRSEVTELRRVLQGLEIELQSQLSMKASLENSLEETKGRYCMQLSQIQGLIGSVEEQLAQLRCEMEQQSQEYQILLDVKTRLEQEIATYRRLLEGEDAHLSSQQASGQSYSSREVFTSSSSSSSRQTRPILKEQSSSSFSQGQSS

SEQ ID NO: 23 - DNA, 13302 bp, Homo sapiens
gaagcgcctgtgctctgccgagactgccgtgcccattgctcgcctcggtcgccgccgctttagccgcctccgggggagcggccgcctattgtctttctccgcggcgaaggtgaagagttgtcccagctcggcccgcgggggagccccgggagccgcacgtgtcctgggtcatgaaacttaatccacagcaagctcccttatatggtgattgtgttgttacagtgctgcttgctgaagaggacaaagctgaagatgatgtagtgttttacttggtatttttgggttccaccctccgtcactgtacaagtactcggaaggtcagttctgatacattggagaccattgctcctggtcatgattgttgtgaaacagtgaaggtgcagctctgtgcttccaaagagggccttcccgtgtttgtggtggctgaagaagactttcatttcgtccaggatgaagcgtatgatgcagctcaattcctagcaaccagtgctggaaatcagcaggctttgaactttacccgttttcttgaccagtcaggacccccatctggggatgtgaattcccttgataagaagttggtgctggcattcaggcacctgaagctgcccacggagtggaatgtattggggacagatcagagtttgcatgatgctggcccgcgagagacattgatgcattttgctgtgcggctgggactgctgaggttgacgtggttcctgttgcagaagccaggtggccgcggagctctcagtatccacaaccaggaaggggcgacgcctgtgagcttggccttggagcgaggctatcacaagctgcaccagcttctaaccgaggagaatgctggagaaccagactcctggagcagtttatcctatgaaataccgtatggagactgttctgtgaggcatcatcgagagttggacatctatacattaacctctgagtctgattcacatcatgaacacccatttcctggagacggttgcactggaccaatttttaaacttatgaacatccaacagcaactaatgaaaacaaacctcaagcagatggacagtcttatgcccttaatgatgacagcacaggatccttccagtgccccagagacagatggccagtttcttccctgtgcaccggagcccacggaccctcagcgactttcttcttctgaagagactgagagcactcagtgctgcccagggagccctgttgcacagactgaaagtccctgtgatttgtcaagcatagttgaggaggagaatacagaccgttcctgtaggaagaaaaataaaggcgtggaaagaaaaggggaagaggtggagccagcacctattgtggactctggaactgtatctgatcaagacagctgccttcagagcttgcctgattgtggagtaaagggcacggaaggcctttcgtcctgtggaaacagaaatgaagaaactggaacaaaatcttctggaatgcccacagaccaggagtccctgagcagtggagatgctgtgcttcagagagacttggtcatggagccaggcacagcccagtattcctctggaggtgaactgggaggcatttcaacaacaaatgtcagtaccccagacactgcaggggaaatggaacatgggctcatgaacccagatgccactgtttggaagaatgtgcttcagggaggggaaagtacaaaggaaagatttgagaactctaatattggcacagctggagcctctgacgtgcacgtcacaagtaagcctgtggataaaatcagtgttccaaactgtgcccctgctgccagttccctggatggtaacaaacctgctgagtcttcacttgcatttagtaatgaagaaacctccactgaaaaaacagcagaaacggaaacttcacgaagtcgtgaggagagtgctgatgctccagtagatcagaattctgtggtgattccagctgctgcaaaagacaagatttcagatggattagaaccttatactctcttagcagcaggcataggtgaggcaatgtcaccctcagatttagcccttcttgggctggaagaagatgtaatgccacaccagaactcagaaacaaattcatctcatgctcaaagccaaaagggcaaatcctcacccatttgttctacaactggagacgataaactttgtgcagactctgcatgtcaacagaacacagtgacttctagtggcgatttggttgcaaaactgtgtgataacatagttagcgagtccgaaagcaccacagcaaggcaacccagctcacaagatccacccgatgcctcccactgtgaagacccacaggctcatacagtcacctctgaccctgtaagggatacccaggaacgtgcggatttttgtcctttcaaagtggtggataacaaaggccaacgaaaagatgtgaaactagataaacctttaacaaatatgcttgaggtggtttcacatccacatccagttgtccctaaaatggagaaagaactggtgccagaccaggcagtaatatcagacagtactttctctctggcaaacagtccaggcagtgaatcagtaaccaaggatgacgcactttcttttgtcccctcccagaaagaaaagggaacagcaactcctgaactacatacagctacagattatagagatggcccagatggaaattcgaatgagcctgatacgcggccactagaagacagggcagtaggcctgtccacatcctccactgctgcagagcttcagcacgggatggggaataccagtctcacaggacttggtggagagcatgagggtcccgcccctccagcaatcccagaagctctgaatatcaaggggaacactgactcttccctgcaaagtgtgggtaaggccactttggctttagattcagttttgactgaagaaggaaaacttctggtggtttcagaaagctctgcagctcaggaacaagataaggataaagcggtgacctgttcctctattaaggaaaatgctctctcttcaggaactttgcaggaagagcagagaacaccacctcctggacaagatactcaacaatttcatgaaaaatcaatctcagctgactgtgccaaggacaaagcacttcagctaagtaattcaccgggtgcatcctctgcctttcttaaggcagaaactgaacataacaaggaagtggccccacaagtctcactgctgactcaaggtggggctgcccagagcctggtgccaccaggagcaagtctggccacagagtcaaggcaggaagccttgggggcagagcacaacagctccgctctgttgccatgtctgttgccagatgggtctgatgggtccgatgctcttaactgcagtcagccttctcctctggatgttggagtgaagaacactcaatcccagggaaaaactagtgcctgtgaggtgagtggagatgtgacggtggatgttacaggggttaatgctctacaaggtatggctgagcccagaagagagaatatatcacacaacacccaagacatcctgattccaaacgtcttgttgagccaagagaagaatgccgttctaggtttgccagtggctctacaggacaaagctgtgactgacccacagggagttggaaccccagagatgatacctcttgattgggagaaagggaagctggagggagcagaccacagctgtaccatgggtgacgctgaggaagcccaaatagacgatgaagcacatcctgtcctactgcagcctgttgccaaggagctccccacagacatggagctctcagcccatgatgatggggccccagctggtgtgagggaagtcatgcgagccccgccttcaggcagggaaaggagcactccctctctaccttgcatggtctctgcccaggacgcacctctgcctaagggggcagacttgatagaggaggctgccagccgtatagtggatgctgtcatcgaacaagtcaaggccgctggagcactgcttactgagggggaggcctgtcacatgtcactgtccagccctgagttgggtcctctcactaaaggactagagagtgcttttacagaaaaagtgagtactttcccacctggggagagcctaccaatgggcagtactcctgaggaagccacggggagccttgcaggatgttttgctggaagggaggagccagagaagatcattttacctgtccaggggcctgagccagcagcagaaatgccagacgtgaaagctgaagatgaagtggattttagagcaagttcaatttctgaagaagtggctgtagggagcatagctgctacactgaagatgaagcaaggcccaatgacccaggcgataaaccgagaaaactggtgtacaatagagccatgccctgatgcagcatctcttctggcttccaagcagagcccagaatgtgagaacttcctggatgttggactgggcagagagtgtacctcaaaacaaggtgtacttaaaagagaatctgggagtgattctgacctctttcactcacccagtgatgacatggacagcatcatcttcccaaagccagaggaagagcatttggcctgtgatatcaccggatccagttcatccaccgatgacacggcttcactggaccgacattcttctcatggcagtgatgtgtctctctcccagattttaaagccaaacaggtcaagagatcggcaaagccttgatggattctacagccatgggatgggagctgagggtcgagaaagtgagagtgagcctgctgacccaggcgacgtggaggaggaggagatggacagtatcactgaagtgcctgcaaactgctctgtcctaaggagctccatgcgctctctttctcccttccggaggcacagctgggggcctgggaaaaatgcagccagcgatgcagaaatgaaccaccggagtatgagctggtgcccctctggtgtgcagtactctgctggcctgagtgctgactttaattacagaagtttcagtctagaaggcttgacaggaggagctggtgtcggaaacaagccatcctcatctctagaagtaagctctgcaaatgccgaagagctcagacacccattcagtggtgaggaacgggttgactctttggtgtcactttcagaagaggatctggagtcagaccagagagaacataggatgtttgatcagcagatatgtcacagatctaagcagcagggatttaattactgtacatcagccatttcctctccattgacaaaatccatctcattaatgacaatcagccatcctggattggacaattcacggcccttccacagtaccttccacaataccagtgctaatctgactgagagtataacagaagagaactataatttcctgccacatagcccctccaagaaagattctgaatggaagagtggaacaaaagtcagtcgtacattcagctacatcaagaataaaatgtctagcagcaagaagagcaaagaaaaggaaaaagaaaaagataagattaaggagaaggagaaagattctaaagacaaggagaaagataagaagactgtcaacgggcacactttcagttccattcctgttgtgggtcccatcagctgtagccagtgtatgaagcccttcaccaacaaagatgcctatacttgtgcaaattgcagtgcttttgtccacaaaggctgccgagaaagtctagcctcctgtgcaaaggtcaaaatgaagcagcccaaagggagccttcaggcacatgacacatcatcactgcccacggtcattatgagaaacaagccctcacagcccaaggagcgtcctcggtccgcagtcctcctggtggatgaaaccgctaccaccccaatatttgccaatagacgatcccagcagagtgtctcgctctccaaaagtgtctccatacagaacattactggagttggcaatgatgagaacatgtcaaacacctggaaattcctgtctcattcaacagactcactaaataaaatcagcaaggtcaatgagtcaacagaatcacttactgatgagggagtaggtacagacatgaatgaaggacaactactgggagactttgagattgagtccaaacagctggaagcagagtcttggagtcggataatagacagcaagtttctaaaacagcaaaagaaagatgtggtcaaacggcaagaagtaatatatgagttgatgcagacagagtttcatcatgtccgcactctcaagatcatgagtggtgtgtacagccaggggatgatggcggatctgctttttgagcagcagatggtagaaaagctgttcccctgtttggatgagctgatcagtatccatagccaattcttccagaggattctggagcggaagaaggagtctctggtggataaaagtgaaaagaactttctcatcaagaggataggggatgtgcttgtaaatcagttttcaggtgagaatgcagaacgtttaaagaagacatatggcaagttttgtgggcaacataaccagtctgtaaactacttcaaagacctttatgccaaggataagcgttttcaagcctttgtaaagaagaagatgagcagttcagttgttagaaggcttggaattccagagtgcatattgcttgtaactcagcggattaccaagtacccagttttattccaaagaatattgcagtgtaccaaagacaatgaagtggagcaggaagatctagcacagtccttgagcctggtgaaggatgtgattggagctgtagacagcaaagtggcaagttatgaaaagaaagtgcgtctcaatgagatttatacaaagacagatagcaagtcaatcatgaggatgaagagtggtcagatgtttgccaaggaagatttgaaacggaagaagcttgtacgtgatgggagtgtgtttctgaagaatgcagcaggaaggttgaaagaggttcaagcagttcttctcactgacattttagttttccttcaagaaaaagaccagaagtacatctttgcatcattggaccagaagtcaacagtgatctctttaaagaagctgattgtgagagaagtggcacatgaggagaaaggtttattcctgatcagcatggggatgacagatccagagatggtagaagtccatgccagctccaaagaggaacgaaacagctggattcagatcattcaggacacaatcaacaccctgaacagagatgaagatgaaggaattcctagtgagaatgaggaagaaaagaaaatgttggacaccagagcccgagaattaaaagaacaacttcaccagaaggaccaaaaaatcctactcttgttggaagagaaggagatgattttccgggacatggctgagtgcagcacccctctcccagaggattgctccccaacacatagccctagagttctcttccgctccaacacagaagaggctctcaaaggaggacctttaatgaaaagtgcaataaatgaggtggagatccttcagggtttggtgagtggaaatctgggaggcacacttgggccgactgtcagcagccccattgagcaagatgtggtcggtcccgtttccctgccccggagagcagagacctttggaggatttgacagccatcagatgaatgcttcaaaaggaggcgagaaggaagagggagatgatggccaagatcttaggagaacggaatcagatagtggcctaaaaaagggtggaaatgctaacctggtatttatgcttaaaagaaacagtgagcaggttgtccagagcgttgttcatctctacgagctcctcagcgctctgcagggtgtggtgctgcagcaggacagctacattgaggaccagaaactggtgctgagcgagagggcgctcactcgcagcttgtcccgcccgagctccctcattgagcaggagaagcagcgcagcctggagaagcagcgccaggacctggccaacctgcagaagcagcaggcccagtacctcgaggagaagcgcaggcgcgagcgtgagtgggaagctcgtgagagggagctgcgggagcgggaggccctcctggcccagcgcgaggaggaggtgcagcaggggcagcaggacctggaaaaggagcgggaggagctccagcagaagaagggcacataccagtatgacctggagcgactgcgtgctgcccagaaacagcttgagagggaacaggagcagctgcgccgggaggcagagcggctcagccagcggcagacagaacgggacctgtgtcaggtttcccatccacataccaagctgatgaggatcccatcgttcttccccagtcctgaggagcccccctcgccatctgcaccttccatagccaaatcagggtcattggactcagaactttcagtgtccccaaaaaggaacagcatctctcggacacacaaagataaggggccttttcacatactgagttcaaccagccagacaaacaaaggaccagaagggcagagccaggcccctgcgtccacctctgcctctacccgcctgtttgggttaacaaagccaaaggaaaagaaggagaaaaaaaagaagaacaaaaccagccgctctcagcccggtgatggtcccgcgtcagaagtatcagcagagggtgaagagatcttctgctgaccctcttcctctctgctgaggcagctgcctcctgatcctggccagcccacctctcctgctgtccccgcgtgcacaagtctcttacactggacgcccactgctcctcagcgtccagtcctcctgggcggccccaggtcctggacaataagcaacagatgatattgagtgtcgggtggggaaggaggcccagactctgcttcggccatgatttgtgactgcccaggactctcaggttgggctggccctactcaggattacactgaaagtaatggcctcgtaagtacaggtgatggttttggacacgtcaggaattcctaaaggctgaaagagtgtatccaagtaaggtctgaacctccgaatgccttttatttgggggaacacaaaaccaaacagcagatgttttggacttgatctgtgtacgtacatggggacctgtctgcatatacacacggggaatgccagaagaaggcccagtctgcaccaggcgtctggtcaacttagcacaagggcagtgcctggacggacccggagcccccgcatatcagcagttcacccagtactcctcagagactggtttccctctaaacccatcccgggcacataccacccgtgttttgcatgtatttctcatttcattttagggatgacaaacatttgtgaaaccagtgagagaaggcttgatgtgtataaaagacgtgatgtgcaccacctcgatctcggtgtttcaggcactaaagcaacaaaacaacccatagtatctcattctgtcatcagatccagaagaaatatcctggttttccagcatgtttacccacatgttttggccatggataaagtgaagaggcctactcaccattatccctgcagcgtgacaccttttgattgtcactgaccactcagaaggggccacggcctcctggctgtgttcctgagcccccgtcgtgcctctcccagacagcagctgtctggcccttgctgggtgagggcacaccactgccaggggtcagcctcgcacccaggccaggcagaagctgtgctctgaagctaggacagctggctgagaagtgggttcaggcgaagggtgaagccatgtgtagcagttcctgccagtgcagatctggagaggagctggcccggaaggcgtggttgtgaaagcgcccttcttatgttaggaggccttggcaaaattggatttcttcaaaaatacatgtaaaggtctgttgttgaattgtactctgcccctggaagcagatacagatggctgcctgctgctcggctttgcttttgcttttcccaccgtgttttcatctttgttcacttgaggctttccccagctggtgtgtgcaggacagttcatggtaatgttgccctctgaggccccgtacaccagaagggaggccctggaaaattttgtgcttccaacgtggccttcaattcttgcttttttgcccctcggaagcatggggcttttgagcacacttaaaaaaagaaaaatctgtaacttggtgcttattgatgaattgcaagctggccttgcagatggagatatttatctttcagtttatttgaaagaggtctggtttaaaatttgtagcctacatttgttttatttattgtatttgtgtgtttgtgtttgtttttttttaagggtgagccaggtctagcccaacagtctaaactatccagtcaataccgagtgaagtggcagccagcactgttcactctgtgtcttttgaagtgccttgaaggcccagatgaaattttaaagggagggggtccatgtccttccctcccccaccccgcctcattctttaatcaaaggatgtcttctcccttgtttgagaatgaagaaactcgccacctctgacctacctttgcctttttctgtcatggagaatactcacccttcagaaacagaccaaaggccaaaacctgctgatttttctattgaaaatatgtccccttgcaaagaccctaaacaaaaagttaagtttctttctttcacctatttgtacaactccaagttacagctgaatctgtcgtgactttcctgagatctacccggggcttggctgtctgttctgggcactggctccgagttcccctcctgggatttgcaggagggcagtactgaacctgcattcttctccttgtaaatgtaggccgggtgcccctgttctccgggtttggaacaatacgaggttggtgctgatgggatttacttgcgtacgtgctcttcacaaaaacaccgtggatgctgaagttagagcacgtcgccacagagcttgacatcaatgttagagggtctcttactccccgcccagctgtgatgtttcatctgctttggttgttttggtggtcttttttaaaaatagagatttcacatctgcccagaccccactcaaaacgatttggtcaggttctggttggacaagtttaaaatcaaagtagtgcccggaattccctcaaaccacccaacttcatccaggaatacagtctgcagtgcagcaacagaaccgcttaccaagaactgtgcttacatacctttgtcatctctcttccccccttggaagttgtcctcagggggatttgttcctgtcctggggatttacctgggatggtggctgcctgtgcttttgctcatggccttgacagtgctctagttgctggatctaatggcctgtcttggtttctatcacatgagaaggggttgtttttttggggtgactcggactgaattccccatactgtttccacgccgggacaccatgttctccatcaagctaaagaaatcacgtgcctgaaactgtgcttaagttttgggggaaagatggagttcctatccagagcccccagatttccagaatcgagtgagcttcctggaaggagactgcgtcttctctcaattccagtcatctcagtcgttgtcgttaggtgacatgtgcactttaaatgctctcatcggttggcttcattttcaagacaatcaaatgtattgactgtgttttcttcttagaaaatggagagggttaaaaacatgcaaactgccactttcaacctttgccagtattccctctacccccgtgagagctatctggggggaagaatccttaccaaggtttttttggaaaggtacgaatcttaacttttttccccttctgtgtctcagggtaatactattcagagtcgcccctttgctcattttctcccgtatttgttaccttcctgaggcctcagtattagtcgtgagcacaaagttttgagacctttggcgttgtttcttgatgtgggaggggaggtgttagtgcatgcaagggttgaactagatagaccctgccttagtagagggtgggactataaccttagaggccagaacttgatccagaagttgctgtccacagaagtgctttctatttcatcatttttgtttctagggctctttttctgtagccaggtcttcccaaggattttagtatttgcattggagttgaggtttactctaatgatggtggcccagctgtgcccagaggacagccaggcaggccctgggagggagtttagaaagacagtcctggtgaatgggcttcaagtggtcacaaagagggtggctgtgaggtgaccccagacactgcagaacgatgtgcaccctctgcgttttggatgtccttggaatgtgggagcctagaaataaccctgtggatggaattggggcagcggctgctggagatctgtgtgccttgccttccttcagcaggaccgtctaggtgcgcagccacctatggatgcgtcccagccagccccgtcgctctcgtccatcctcagagacaaagaagagggcagggagtttgggcttggttttgaactttcctttcaatgtagcaaagcattcctagttaaccagagccttggaatctactgcctgctggccaggctttaaaatgaaaagtgttttaatgctgccataaaagggaggcgggggggaggaagggaaaataaaggcatctttccaagtactcatctaatttaattgtcaaaagattgataggccatgaattacttctccatctcactaagggttaaaggcgtgcaaccccccactggctgtgtcccctgccaccgaagtgagtgacctgccctacaaccaggtgggaccacctgtgctgcagtccggaggggcttctgcaggaagcactcaccccccacaccttccccggcctgagcttcccctacctttcgtcaccacctgagggcatgagcacaggccatggggcgtgcctggtgagtctgcctgtggttcaggcttagcctgtggtctcctgtgtgctgctgcccgcatgggatgcgcaggggaggcgtggggatccgcaggagggtggttgggatacaccggatacctctgctctcattgcttgtttgcaaatgctctatggacatttgtgtgctaaatcctattaaataaaaaagacgggttaaaacccagatgctgtatattcatttgtaattatgtataaagtgaagcagttttaaactgtaaagatttttttcagtgtgttttctcgaattttgccacaacatactggcttcgtattttatttatctttctttctagttaccagcttcagacccttgtaaagtctccctcagccctttcaaaaaataataaatttcctgtgaagtt

SEQ ID NO: 24 - DNA, 3115 bp, Homo sapiens
tgggtagagtccagcctagtgagagctgagtgaaggggctggccatgcctgagacaaaaagtcaaatgagacaatggacgtgtcaatgacttgaaaaaaagtcacatccagcaaatgcagggtcacatgaaatatgggcctcctggaatccctacagtggatggagactggctcataccttgccagatccctctctcagttccagccttctggacaaggcctgggctaagaggagctgattcgttatctcttcacccactgccctctcagtatcaccagtcccaaagacaggatacgtccctgtaacccaatctctcggttgattgatagcagaacagctcttgttggtctgagaaggcaggataagtgaccacatatttatgccactacctccaccagggagagtccttctccacaggcttgataaattcaatcaccaactgtgctgtcgtccctgactctgctactcccgttcttcctgctttcctgctccgtatctcagtctgcactgaccccagggctgggctgacatcaagatgggagcccagcccacgggctttataaacacccaagaaccgtttcagatcttctctgtgctgatgcaggtagttttaaatttttctcagttccagtgatagaaaacccacacaatacatcctctgccagtcttaatagaatatcagaggtaagaggggcctcagagaagctctgacgcagtgctgctggggaagggaagtgactaaccccgggtcagcctgccatttagggaaagagctgaggttcttacccttgttgcatgctgccacctctccttagccagtgctcttgtacatccacacagcaccctaaggagccatagtcaccatcaaagactcaaccctaaggcccttcaagatctcaaagtgccttctgaagcatcagagattaaatattgttcaaactaatagttattgctgtggcttttaattttatctttggaagatagctatatggtaactcatcattaaccagaacacctctcccctcaaattccgtgaccaagttgtgcagcttgagcaaatgccgaaagagggtattatgggtgggtggtgtgggcttgcaaatacaagcttggaggtgagacatggccagacatgactcctgcttccccttaggaagtaaatcttacttatggttgtgaactgcttggagtccaggatgcccagatgtgaggggcagatgaagggaatgttgctggaaaggtgccttttaaggctgctgagaatttctggactgtgtcctgatggacgcagcaccatcaaagcccagaatttctgaaaacggtgacaaggttaacataaggacaacaaatactccaccctgtcatggtatgtgaggtgtgggtgtggcggtttctgtgtacgtttgctcatacacgcacatccaaaagcctgtgcctcatccctggccatgggtgaggacttggtctgtcacggctgatgaggactcccacaaccggccaagttatgtcttattatacacccccagaaagagagaaagctgccttctggaggactgattccacatgctatattcagctgagttgatttctgtgtctatttcaacccataacctgaagaatgatcaccttattccttattcattaattttcttgattaatagggaaacttgggaatagctataaagtaaaacttgggtggaacctggggccctggcatcacacaagtgtgattaggatggtcaaggtcatcaggagtacagcctattatattcccacatcctgagaaaggtcatttctcccacacacgacaaagtcacagacatcctgcacctgccactaggcatcctcatcctactgacatgcccatttctccagttttcttaatctgagactcccttcccttgttttttaaagataccgtgcttctccacatcctcatccttcaaggagcatattttgctcttaggatggtctttgggattcaagaatagaataataaatccaaacttggtcattcccattttgaagagatgcaagagggcccagtgaggacatccgcctccctgaaagtggtgctagacagagctgaggtcattgtatctgtgtatccacataggatttctcttaattcagcttgaattgatggggagggaggtaagagtagggtcagagttactcatcccttttcaaagaattgtgggtggaagtttgtaaaggccattcatttgattttcaaaatcaaagcgacagctctacttccacttggccttagatctctgctataccctgccatagccttgatgccactgggcacaagccacctgccaaatacaggagtggcctctcccagcctggcatgataggggggtctgtgccctcagatgtgttgacagctgctcttctgaattgccacacctgtgctacacttggaattctgtgctctgactctgcagggtaggaccacgtgccatctcacacagaggtcaaccgatgagcccactcactcgtacatgccttcttccacagtgggaagcatgatctggcaggggccgccctgtaggctggggatgggctgctgtgtgaatgttgacgttcgtttcatggagaaaggggaggtgaaagattgaagagcaggttcctgtcaatgttctgagttcgagctggaggtgtagattgaatagtctacatggtctgtgagtgtgtgagatgaacccttccatcctttgacacctggttgtatgtgtaggctaagaaggaaggaccctcctgtcagtgtgcaaagctgtaatctcatggactagaggagagggggccaaggggatggacaggagaagtcatgcagaatctaagcaggaatgcagatagaacacatctaggctcttttccccaggagagtgatgatggagcatatagatctggctcaaattcagcctccatcacttaccagtcaggaaccctggcgatatcactttaactttctgaacctcagagtcttcacctataagacggggaaaataataccaccctttcaagattgttgagataaataagtgatataaaacatgtaaagcttagttctggccacagtgtagctactcaataaatgataatact

SEQ ID NO: 25 - DNA, 1528 bp, Homo sapiens
ggaaggcacaggcctgagaagtctgcggctgagctgggagcaaatcccccaccccctacctgggggacagggcaagtgagacctggtgagggtggctcagcaggaagggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacctggggaaataaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtggaaaaaaaaaaaaaaaaaaaaaaaaaaaaaaaaaaa

SEQ ID NO: 26 - DNA, 1927 bp, Homo sapiens
tgccagcccaagtcggaacttggatcacatcagatcctctcgagctccagcaggagaggcccttcctcgcctggcagcccctgagcggctcagcagggcaccatggcaagatcccttctcctgcccctgcagatcttactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcgtggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaaga

SEQ ID NO: 27 - DNA, 392 bp, Homo sapiens
atatcaaggagtgtcctcttgctcctcttccaccctcagtggatgataatctgaaggagtgtctcctggtccctcttccaccctctcctcttccaccctcagtggatgataatctgaaggactgtctctttgtccctcttccaccctctcctcttccaccctcagtggatgataatctcaagactcctcccttagctactcaggaggccgaggcggaaaaaccacccaaacccaagaggtggagggtggatgaggtggaacaatcaccgaaacccaagaggcggagggcggatgaggtggaacaatcgcccaagcccaagaggcagagggaggccgaggcacaacaattacccaaacccaagaggcggaggttgagtaagctgagaacacgc

SEQ ID NO: 28 - DNA, 944 bp, Homo sapiens
tcaagattaaacgacaaggacagacatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctgacctccttttaccttctgatacctggaaatccctgccctgttcagccccacagctcccaactatttggttcctgctccatggtcgggcctctgacagccactttgaataaaccagacaccgcacatgtgtcttgagaattatttgg

SEQ ID NO: 29 - DNA, 3856 bp, Homo sapiens
aacgccgggcagggcggcgggcgcgctcagtctggcggcggctgccgtgagctgactgacgttccgggaacgccgcagcagcccgcgccgcccgcagcctagccgagccgcgccgcccgggcctcgcccgcccgcctgcccgccatggtgtcatggatcatctccaggctggtggtgcttatatttggcaccctttaccctgcgtattattcctacaaggctgtgaaatcaaaggacattaaggaatatgtcaaatggatgatgtactggattatatttgcacttttcaccacagcagagacattcacagacatcttcctttgttggtttccattctattatgaactaaaaatagcatttgtagcctggctgctgtctccctacacaaaaggctccagcctcctgtacaggaagtttgtacatcccacactatcttcaaaagaaaaggaaatcgatgattgtctggtccaagcaaaagaccgaagttacgatgcccttgtgcacttcgggaagcggggcttgaacgtggccgccacagcggctgtgatggctgcttccaagggacagggtgccttatcggagagactgcggagcttcagcatgcaggacctcaccaccatcaggggagacggcgcccctgctccctcgggccccccaccaccggggtctgggcgggccagcggcaaacacggccagcctaagatgtccaggagtgcttctgagagcgctagcagctcaggcaccgcctagaatccttcgatctcgcttcaggaagaaaagtacctcatcctcggccaccgaaaccacgtgagtgagatgagccaacagcaccggatccacagaatgtttcttctctgccttaaagagctattcactaataacatagaaatccgcaagctgggtgtgctttgagtgtgcagcctcacaaacatggccttttctctctccccttccacttttaaggatttatttttttcccccttttctttattttgctggggagaggctaaagggaaaggtagtaggggcgggggtggtgacctttaagtcttctgaggttggtaattttccacaattggattgtcattatagacagcagtgtgttttttagaaagataagagaatcacccctatgctgctgagatgtacatttgtaatttatctgttgcatacttagtttttagtcctgtaaatgcaaacacagcattttttacaactttctttgttcttggtacttatactttgaactatgatgtacatatttatggcttttggcttttaatataatggacttgcaagggctgccagaggttctgatatgtaagaaaactgcaaaaacaaatatagacaaatattttgattctagagaacgtctcagatgtgcttataaagcttccaaatacaactccagtaagacatccctttccctgcaggagtgtggtctatattctttagatagttgtttagtcaaaagaccagacaagttacaaactaagagaaacaatatttcacaacacagtaaagtgtgatgagaggtcaggggaacatcccagtaaaagagaagagtcacaggaagctcatctcctccctggattctggattaggagcttctgaatcttttccagggataggcaggtagctcactcttggtgcaatttcttgaggatgggaacatgtagagctgctggaaggagtaattctgtgcttgacaaaggacgatttctcctttatcgtgaccagtgctgccgatttcctgacagaggagcttacactctgagcaccttgttttagcgaactctagcaaaacttgtttagcttagcaaaaacaaacacacaaaaaactgagaactctgctgtttcagatatgccataacatacatctgaaacacatgtgtaacaatcaaaatggtgggctctagaatggttttggagctcgagatcttcatgggttagacttgctggtcagacccaggagcacctgtggctcacaccttctgttcccctcctggcctgtgcagaatgtaaacagcagactcatactcaatgggcactacaggccttatcagacgttttatacaagcctggattgcttagtaggggaataaggcattctctgagggggctttccacttagattgagaattttatttgaaaagaatctggtttaaatggcattgtggtccgaggtagctgctctccccactgagagctgagccgaaatataagaataatatatttgtgcttcgagttggtgtttctttcagtgtaatgcatgcagtggtcacaacccagttactcataatatttggattgtatttgttcgtagatatgcccagaagactagagaattagtgttatataccatatagaacttactgtcagtcaactataaacaggcccaattaaaaactgttccattactacgcaaacacatattagaggcctttgctgatgacacattagctggatcttagccaccccagaaagggtttgatttgaagctgattgttgccagatatgcatattggaatcccatctacccatagttcctctgaaggtgattttgtaatttgcaaaagggtataggaaaatatacctaaaagcgaatttgtggctgagaggataaacagaagctgtttgctcatgttctgtgccccacacccaccaatacctaaatctgttaaggaagacagaaaatgttttctttgtgctcattgagtagttccagacagaagaagaatatactctttaaaatgtatttacctgttagttggaagtacccagaattatcagaaacgaatgcaaaaaaaaaaaaaaaaaaaaaaagcttacacagcttcttagcaattttttttttttttgccgaaacaataaattgcctttagcagcagtttaaaatcctatcgtgaacaacctatattttcgccattttacaatggagagttgtgacaagtacaggttatcaagtttgcacttaactatgccaaaaaaagtttgaagcgctctattctcagacatgctgtattattacttctcattcaagattgaaaaatataaaggtatccaaactctgtcttaatgtaaatgtaactatttttccttcaagtgttgactagggagtcggtttctctcttaaagacactcactgtacaactgaaagcagctgtcatatttctggcaaaatgtgtttacgtatctgacaagttgtacatttgtgtatgaactgacataaaatgtgaaagcctgtaagtgtacatgtagtggtgtggtgttctgtctagaggatacaactgaatgtttttaatttgctgacttacagacacaggctgtttacaaaatgctagctggaaagtctgtaatgttcatgtcataacttttagttaattgccattgagcacctgttctgaggaggtgagatgtggacttgtgcttataaactggagagtttagtcataatccctcctggctttgtgtgaatagcttgctcactttgctggcctttgaaatgtgttctccgtgataagctatccatgtgtttgtgataagagtgcttgtcaaccatgaccatctttgagccttcctagtcctccacctggcacagtatttgaaatggcaaaggatgtgcttcatcctctaacaaacagtgtacactcccagagctgatattctggattgtgactgtgcacatttcctctagttcatgtctgtagtccctatagaatgatctgtaataaaatagtatactggactgtgcatcaaagggatgtaaaattacagtattccaaaggttgaagttctgctgttttgttataatgcctgatacacatcttgaataaagtcttaacatttttctttt

SEQ ID NO: 30 - protein, 2817 aa, Homo sapiens
MKLNPQQAPLYGDCVVTVLLAEEDKAEDDVVFYLVFLGSTLRHCTSTRKVSSDTLETIAPGHDCCETVKVQLCASKEGLPVFVVAEEDFHFVQDEAYDAAQFLATSAGNQQALNFTRFLDQSGPPSGDVNSLDKKLVLAFRHLKLPTEWNVLGTDQSLHDAGPRETLMHFAVRLGLLRLTWFLLQKPGGRGALSIHNQEGATPVSLALERGYHKLHQLLTEENAGEPDSWSSLSYEIPYGDCSVRHHRELDIYTLTSESDSHHEHPFPGDGCTGPIFKLMNIQQQLMKTNLKQMDSLMPLMMTAQDPSSAPETDGQFLPCAPEPTDPQRLSSSEETESTQCCPGSPVAQTESPCDLSSIVEEENTDRSCRKKNKGVERKGEEVEPAPIVDSGTVSDQDSCLQSLPDCGVKGTEGLSSCGNRNEETGTKSSGMPTDQESLSSGDAVLQRDLVMEPGTAQYSSGGELGGISTTNVSTPDTAGEMEHGLMNPDATVWKNVLQGGESTKERFENSNIGTAGASDVHVTSKPVDKISVPNCAPAASSLDGNKPAESSLAFSNEETSTEKTAETETSRSREESADAPVDQNSVVIPAAAKDKISDGLEPYTLLAAGIGEAMSPSDLALLGLEEDVMPHQNSETNSSHAQSQKGKSSPICSTTGDDKLCADSACQQNTVTSSGDLVAKLCDNIVSESESTTARQPSSQDPPDASHCEDPQAHTVTSDPVRDTQERADFCPFKVVDNKGQRKDVKLDKPLTNMLEVVSHPHPVVPKMEKELVPDQAVISDSTFSLANSPGSESVTKDDALSFVPSQKEKGTATPELHTATDYRDGPDGNSNEPDTRPLEDRAVGLSTSSTAAELQHGMGNTSLTGLGGEHEGPAPPAIPEALNIKGNTDSSLQSVGKATLALDSVLTEEGKLLVVSESSAAQEQDKDKAVTCSSIKENALSSGTLQEEQRTPPPGQDTQQFHEKSISADCAKDKALQLSNSPGASSAFLKAETEHNKEVAPQVSLLTQGGAAQSLVPPGASLATESRQEALGAEHNSSALLPCLLPDGSDGSDALNCSQPSPLDVGVKNTQSQGKTSACEVSGDVTVDVTGVNALQGMAEPRRENISHNTQDILIPNVLLSQEKNAVLGLPVALQDKAVTDPQGVGTPEMIPLDWEKGKLEGADHSCTMGDAEEAQIDDEAHPVLLQPVAKELPTDMELSAHDDGAPAGVREVMRAPPSGRERSTPSLPCMVSAQDAPLPKGADLIEEAASRIVDAVIEQVKAAGALLTEGEACHMSLSSPELGPLTKGLESAFTEKVSTFPPGESLPMGSTPEEATGSLAGCFAGREEPEKIILPVQGPEPAAEMPDVKAEDEVDFRASSISEEVAVGSIAATLKMKQGPMTQAINRENWCTIEPCPDAASLLASKQSPECENFLDVGLGRECTSKQGVLKRESGSDSDLFHSPSDDMDSIIFPKPEEEHLACDITGSSSSTDDTASLDRHSSHGSDVSLSQILKPNRSRDRQSLDGFYSHGMGAEGRESESEPADPGDVEEEEMDSITEVPANCSVLRSSMRSLSPFRRHSWGPGKNAASDAEMNHRSMSWCPSGVQYSAGLSADFNYRSFSLEGLTGGAGVGNKPSSSLEVSSANAEELRHPFSGEERVDSLVSLSEEDLESDQREHRMFDQQICHRSKQQGFNYCTSAISSPLTKSISLMTISHPGLDNSRPFHSTFHNTSANLTESITEENYNFLPHSPSKKDSEWKSGTKVSRTFSYIKNKMSSSKKSKEKEKEKDKIKEKEKDSKDKEKDKKTVNGHTFSSIPVVGPISCSQCMKPFTNKDAYTCANCSAFVHKGCRESLASCAKVKMKQPKGSLQAHDTSSLPTVIMRNKPSQPKERPRSAVLLVDETATTPIFANRRSQQSVSLSKSVSIQNITGVGNDENMSNTWKFLSHSTDSLNKISKVNESTESLTDEGVGTDMNEGQLLGDFEIESKQLEAESWSRIIDSKFLKQQKKDVVKRQEVIYELMQTEFHHVRTLKIMSGVYSQGMMADLLFEQQMVEKLFPCLDELISIHSQFFQRILERKKESLVDKSEKNFLIKRIGDVLVNQFSGENAERLKKTYGKFCGQHNQSVNYFKDLYAKDKRFQAFVKKKMSSSVVRRLGIPECILLVTQRITKYPVLFQRILQCTKDNEVEQEDLAQSLSLVKDVIGAVDSKVASYEKKVRLNEIYTKTDSKSIMRMKSGQMFAKEDLKRKKLVRDGSVFLKNAAGRLKEVQAVLLTDILVFLQEKDQKYIFASLDQKSTVISLKKLIVREVAHEEKGLFLISMGMTDPEMVEVHASSKEERNSWIQIIQDTINTLNRDEDEGIPSENEEEKKMLDTRARELKEQLHQKDQKILLLLEEKEMIFRDMAECSTPLPEDCSPTHSPRVLFRSNTEEALKGGPLMKSAINEVEILQGLVSGNLGGTLGPTVSSPIEQDVVGPVSLPRRAETFGGFDSHQMNASKGGEKEEGDDGQDLRRTESDSGLKKGGNANLVFMLKRNSEQVVQSVVHLYELLSALQGVVLQQDSYIEDQKLVLSERALTRSLSRPSSLIEQEKQRSLEKQRQDLANLQKQQAQYLEEKRRREREWEARERELREREALLAQREEEVQQGQQDLEKEREELQQKKGTYQYDLERLRAAQKQLEREQEQLRREAERLSQRQTERDLCQVSHPHTKLMRIPSFFPSPEEPPSPSAPSIAKSGSLDSELSVSPKRNSISRTHKDKGPFHILSSTSQTNKGPEGQSQAPASTSASTRLFGLTKPKEKKEKKKKNKTSRSQPGDGPASEVSAEGEEIFC

SEQ ID NO: 31 - protein, 293 aa, Homo sapiens
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 32 - protein, 253 aa, Homo sapiens
MARSLLLPLQILLLSLALETAGEEAQGDKIIDGAPCARGSHPWQVALLSGNQLHCGGVLVNERWVLTAAHCKMNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 33 - protein, 130 aa, Homo sapiens
IKECPLAPLPPSVDDNLKECLLVPLPPSPLPPSVDDNLKDCLFVPLPPSPLPPSVDDNLKTPPLATQEAEAEKPPKPKRWRVDEVEQSPKPKRRRADEVEQSPKPKRQREAEAQQLPKPKRRRLSKLRTR

SEQ ID NO: 34 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 35 - protein, 201 aa, Homo sapiens
MVSWIISRLVVLIFGTLYPAYYSYKAVKSKDIKEYVKWMMYWIIFALFTTAETFTDIFLCWFPFYYELKIAFVAWLLSPYTKGSSLLYRKFVHPTLSSKEKEIDDCLVQAKDRSYDALVHFGKRGLNVAATAAVMAASKGQGALSERLRSFSMQDLTTIRGDGAPAPSGPPPPGSGRASGKHGQPKMSRSASESASSSGTA